(19) United States
(12) Patent Application Publication
(10) Pub. No.: US 2002/0016289 A1
(43) Pub. Date: Feb. 7, 2002

(54) METHODS FOR TREATMENT AND PREVENTION OF HELICOBACTER PYLORI INFECTION USING LACTOFERRIN

(76) Inventors: ORLA M. CONNEELY, HOUSTON, TX (US); PAULINE P. WARD, HOUSTON, TX (US); DENIS R. HEADON, HOUSTON, TX (US)

Correspondence Address: HOWREY SIMON ARNOLD & WHITE, LLP, BOX 34, 301 RAVENSWOOD AVE., MENLO PARK, CA, 94025, US

(21) Appl. No.: 09/257,905

(22) Filed: Feb. 25, 1999

Related U.S. Application Data
(63) Continuation-in-part of application No. 08/457,469, filed on Jun. 1, 1995, now abandoned.

(51) Int. Cl. A61K 38/00; A61K 38/16; C07K 1/00; C07K 2/00; C07K 14/00; C07K 17/00

(52) U.S. Cl. 514/8; 514/12; 530/365; 530/395

(57) ABSTRACT

The present invention is directed to methods for using lactoferrin as a therapeutic and/or prophylactic compound to treat and/or prevent infections caused by enteropathogens such as H. pylori. The present invention is directed to the treatment or prevention of diseases and disorders resulting from infection by enteropathogens such as H. pylori including histological gastritis, functional dyspepsia, duodenal ulcers, gastric ulcers, gastric cancer, chronic renal failure, HIV, pernicious anemia, Zollinger-Ellison syndrome and colonic polyps. The present invention is further directed to novel formulations and compositions comprising lactoferrin and pharmaceutically acceptable carriers, excipients and/or adjunct companion therapies such as one or more antibiotics.

### FIELD OF THE INVENTION

The present invention is in the field of molecular biology, biochemistry and medicine. Specifically, the present invention provides methods for treating bacterial infections, especially Helicobacter pylori infections using lactoferrin alone or in combination with another agent such as an antibiotic.

### BACKGROUND OF THE INVENTION

Helicobacter pylori is an etiologic agent of gastric and peptic ulcer disease which infects over 50% of the human adult population. Diseases such as histological gastritis, functional dyspepsia, duodenal ulcers, gastric ulcers, gastric cancer, chronic renal failure, HIV, pernicious anemia, Zollinger-Ellison syndrome and colonic polyps have been associated with H. pylori infection. The present invention is directed to methods for treating and/or preventing infection with such organisms as H. pylori and related diseases resulting from infections with the same using lactoferrin alone or in combination with an antibiotic. The present invention is further directed to novel formulations and compositions comprising lactoferrin and pharmaceutically acceptable carriers, excipients and/or adjunct companion therapies.

Helicobacter is a member of the superfamily VI of Gram-negative bacteria. Vandamme et al., Int. J. Syst. Bacteriol., 1991, 41:88-103. It is a microaerobe having an asaccharolytic metabolism, a membrane bound urease and an aptitude for living in mucus. There are at least 9 known species of Helicobacter.

H. pylori (formerly known as Campylobacter pylori) is a motile S-shaped bacterium about 3 μm long and 0.5-1.0 μm wide. The genome sizes of H. pylori isolates have been determined at about 1.60-1.72 Mb (average 1.67 Mb). Lipopolysaccharides from the bacterium resemble those of the smooth-type Enterobacteriaceae. Surface receptors appear to fall into three classes of hemagglutination patterns and both strain specific and type specific surface receptors are present. Clinical isolates of H. pylori which have a vacuolating cytotoxic protein of 78 KD and an immunodominant cytotoxin associated antigen of approximately 120-128 KD (Type I isolates) have been shown to be the pathogenic form of the organism. Isolates which lack these proteins (Type II) are not infective. Eaton et al., Infect. Immun., 1989, 57:1119-1125, Marchetti et al., Science 1995, 267:1655-1658. The phenotypic appearance of the organism is homogeneous under adequate growth conditions, but as growth conditions deteriorate, H. pylori is believed to evolve from motile forms into coccidial forms. For example, comparison of H. pylori isolated from different locations in the stomach/duodenum has revealed a heterogeneity in size. One reason that patients with H. pylori infection may relapse following treatment might be due to the transformation of the organism from the motile form to the coccoid form, thereby becoming insensitive to the current treatment.

Several enzymes (urease, oxidase, catalase, alkaline phosphatase, y-glutamyl transferase, DNase and various esterases) are used as obligate markers in the biochemical identification of Helicobacter species. Marker enzyme negative mutants have been identified during culture, but are considered rare. The in vitro antimicrobial susceptibility of Heliobacter species is similar across isolates and antibiotic resistant strains are readily produced.

H. pylori infections are typically acquired in childhood although the route of infection, either oral-oral or oral-fecal transmission, has not yet been clarified. Lee et al., Epidemiol. Infect., 1991, 107:99-109. Although treatment regimens do exist to eradicate H. pylori, the subsequent outbreak of infection in a patient is typically the result of inadequate treatment rather than reinfection. The frequency of continued infection may be the result of whether a patient harbors H. pylori reservoirs, such as in the case of the dental plaque of H. pylori gastritis patients. Nguyen et al., J. Clin. Microbiol., 1993, 31:783-787 or incomplete eradication during an earlier antibiotic treatment regimen. Continued infection may also result from the considerable diversity reported between H. pylori strains (each strain can be differentiated from another). This diversity was evidenced by DNA-DNA hybridization techniques that show differences between strains of H. pylori isolated from patients with duodenal ulcers and asymptomatic patients. Yoshimura et al., Dig. Dis. Sci., 1993, 38:1128-1131.

In general, the probability of H. pylori infection is directly proportional to age and inversely related to socioeconomic condition. For example, in the metropolitan area of Houston, Tex., 52% of 485 asymptomatic individuals between 15 and 80 years old tested positive for H. pylori. Graham et al., Digest. Dis. and Sci., 1991, 36:1084-1088. It is generally accepted that three significant human diseases, active chronic gastritis, peptic ulcer disease and gastric adenocarcinoma, have companion infections by Helicobacter spp., and particularly, H. pylori. In addition, H. pylori has also been implicated in malignant lymphoma of mucosa-associated lymphoid tissue of the stomach (Fox, 1994, Proc. Int. Symp., Amelia Is. Florida (Nov. 3-6, 1993), Kluwer Acad. Pub. pp. 3-27), chronic renal failure, HIV, pernicious anemia, Zollinger-Ellison syndrome and choleric polyps. Lambert and Kim, “Prevalence/Disease Correlates of H. Pylori,” Helicobacter pylori—Basic Mechanisms to Clinical Cure, 1994, Kluwer Acad. Publ., pp. 95-112.

H. pylori infection occurs on the luminal surface of the mucosas, both in the mucus and on the epithelial surface, and within the gastric pits. The intragastric environment is hostile to antibiotic therapy, because the medication must diffuse through, or into, a thick mucus layer where the pH may vary from 1 to 7. Antibiotics which are not active in an acid environment have therefore proven to be ineffective. Graham et al., Am. J. Gastroent., 1989, 84:233-238.

The antimicrobial therapies developed to treat Helicobacter sp. are consequently complex with successful eradication directly correlated with duration of treatment and with patient compliance and have limited effectiveness. Current treatment of H. pylori includes: (1) “Triple Therapy,” as described in Al-Assi et al., 1994, Am J. Gastroent., 89:1203-1205, comprising administration of tetracycline HCI (or alternatively amoxicillin) (500 mg 4 times daily), bismuth sybsalicylate tablets (2 tablets, 4 times daily) and metronidazole (250 mg, 3 times daily) (or alternatively, clarithromycin (500 mg, q.i.d.)); and (2) “Double Therapy,” as also described in Al-Assi et al., supra, comprising administration of amoxicillin (750 mg, 3 times daily) and metronidazole (500 mg, 3 times daily) (or alternatively clarithromycin (500 mg, q.i.d.)). For each of these treatment regimens, drugs are given for 14 days with meals, or with meals and at bedtime. The current drug regimens generally also include the administration of an antisecretory drug for ulcer healing. Peura et al., Gastroent., 1994, 89:1137-1139. These “Triple” and “Double” Therapies, as well as, dual agent therapies using proton-pump inhibitors such as omeprazole which also kills H. pylori (Bayerdorffer et al., Abstract, Gastroent., 1992, 102:A38) or Lansoprazole (Iwahi et al., Antimicrob. Agens. Chemoth., 1991, 35:490-496) together with a second antimicrobial (such as amoxicillin (1 g, b.i.d.) or clarithromycin (500 mg, q.i.d.)) have limited effectiveness. Even in the case of bismuth, an effective and inexpensive drug which reduces the rate of development of antibiotic resistance, treatment of H. pylori infection is still considered to be an art rather than a science. Peura and Graham, J. Am. Gastroent., 1994, 89:1137-1139. Moreover, patient compliance with the current therapies is sporadic. Because of the complexity of the treatment regimen and the adverse side effects associated with the antimicrobials used to date, there exists a high non-compliance component within patient populations.

Finally, inadequate or inappropriate treatment regimens lead to the emergence of drug resistant strains which likely result in more frequent side effects. Similarly, the increasing use of anti-Helicobacter regimens also results in an increase in the number of drug resistant H. pylori strains. See, Peura et al., J. Am. Gastroent., 1994, 89:1137-1139. Alternative therapies which do not contribute to or promote generation of antimicrobial resistant strains are therefore desirable.

The glycoprotein, lactoferrin, is a member of the iron-binding family of proteins referred to as transferrins. It is present in nature in the gastric mucosal secretions. In humans, as well as other mammals, lactoferrin is found in high concentrations in milk, with lesser concentrations in plasma, tears, saliva, other exocrine secretions and in polymorphonuclear neutrophils. Masson et al., J. Exp. Med., 1969, 130:643-658. The native lactoferrin molecule is a single chain, 78 kilodalton, glycosylated protein with two (2) major lobes. Each lobe of the molecule binds one (1) atom of ferric iron and one (1) molecule of obligatory carbonate binding one anion, usually bicarbonate. The amino acid sequence (U.S. Pat. No. 5,571,691) 3-dimensional, X-ray crystallographic structure (Anderson et al., J. Mol. Biol., 1989, 209:711-734), and glycosylation patterns (Spik et al., Dur. J. Biochem., 1982, 121:413-419) of lactoferrin have been disclosed previously.

The cDNA sequences for human lactoferrin were disclosed in U.S. Pat. No. 5,571,691, which is incorporated herein by reference. The patent further discloses the use of such cDNA sequences to produce human lactoferrin in a variety of different organisms, including various fungi, such as Saccharomyces cerevisiae, Aspergillus nidulans, Aspergillus oryzae and Aspergillus awamori.

Lactoferrin's antimicrobial properties are supported by both in vitro and in vivo data. For example, it is believed that lactoferrin in human milk is responsible for the low diarrheal incidence observed in breast fed infants compared to formula fed infants because the lactoferrin protein acts to decrease the availability of iron to iron-requiring microorganisms, thereby interfering with such microorganisms' growth and reproduction. Prentice et al. Acta Paediat. Scand., 1989, 78:505-512.

Three different mechanisms, involving at least two separate domains of the protein, contribute to the antimicrobial function. The first is a bacteriostatic function provided by the two iron-binding domains which bind iron with very high affinity thereby depriving bacteria of this essential growth nutrient. Bullen et al., Current Top. Microbiol. Imunol., 1978, 80:1-35; Griffiths et al., Iron and Infection, chapter 5, 1987, pp. 171-209. A similar response to iron withholding has inhibited growth in some fungi. Kirkpatrick et al., J. Inf. Dis., 1971, 124:539-544; Epstein et al., 1984, Rev. Infect. Dis. 61(1):96-106.

The second antimicrobial mechanism is a direct bactericidal domain in a region distinct from the iron binding sites and located at the N-terminus of the protein. Bellamy et al., Biochem. Biophys. Acta., 1992, 1121:130-136. This bactericidal domain induces bacterial membrane permeability changes and causes the release of lipopolysaccharide from the outer membrane of a broad spectrum of bacteria. Arnold et al., Infect. Immun., 1982, 35:792-799; Bellamy et al., 1992, J. Appl. Bacteriol., 73:472-479.

The bactericidal domain of lactoferrin, either as the intact molecule or as specific fragments has an apparently broad spectrum of antimicrobial action. Bellamy et al., Biochem. Biophys. Acta., 1992, 1121:130-136; Bellamy et al., J. Appl. Bacteriol., 1992, 73:472-479.

The third mechanism is believed to be a result of stimulation of systemic immune responses. High affinity lactoferrin receptors have been identified on specific cells of the immune system, such as lymphocytes (Mazurier et al., Eur. J. Biochem., 1989, 179:481-487), monocytes (Birgens et al., 1983, Brit. J. Haematol., 54:383-391), macrophages (van Snich et al., 1976, J. Exp. Med. 144:1568-1580) and platelets (Maneva et al., Int. J. Biochem., 1993, 25:707-712).

Despite the great weight of evidence suggesting the antibacterial effect of lactoferrin, the glycoprotein has been shown, prior to the present invention, to assist rather than inhibit the growth of H. pylori. For example, in 1993 Husson et al., investigated the in vitro activity of native human lactoferrin and other iron containing proteins on the growth of 15 isolates and an ATCC reference strain (#43504) of H. pylori in a study of lactoferrin as an iron chelator/iron donator. Husson et al., Infect. Immun., 1993, 61:2694-2697. Evidence suggested that H. pylori acquired iron directly from lactoferrin using a mechanism in which the host lactoferrin was bound directly to bacterial outer membrane receptors and the iron was removed from the protein for subsequent use by the microbe. Husson et al., supra. See also, Herrington and Sparling, Infect. Immun., (1985) 48:248-251 and Neilands, Annu. Rev. Microbiol., (1982) 36:285-309 regarding Haemophilus influenzae and Neisseria spp. Specifically, Husson et al., reported that H. pylori acquired iron from 10 μM (0.78 mg/ml) 30%-iron-saturated human lactoferrin by direct lactoferrin-organism contact and that this iron was available for use by the organism for growth. As controls, the iron from iron-siderophores enterochelin and pyochelin was not available to H. pylori. Iron was also not available from human transferrin, bovine lactoferrin or hen ovolactoferrin. This evidence strongly indicated that human lactoferrin played a major role in the virulence, rather than control, of H. pylori infections. Additionally, prior to the present invention, lactoferrin has not been shown efficacious against other enteropathogens such as Shigella and Escherichia coli.

### DESCRIPTION OF THE FIGURES

FIG. 1 demonstrates the typical histologic changes including villous blunting with sloughing of epithelial cells, submucosal edema, infiltration of leukocytes, venous congestion, and hemorrhage of ill animals sacrificed 24 hours after infection with E. coli. The degree of inflammation was significantly greater in the M90T than in M90T-rhLF treated animals. These data demonstrate the efficacy of lactoferrin in treating E. coli infections.

FIG. 2 demonstrates that the stomach/body weight ratio was significantly higher in infected mice and was reduced somewhat by lactoferrin alone and the combination of low-dose amoxicillin and lactoferrin, but not significantly reduced by omeprazole and lactoferrin.

FIG. 3 demonstrates that gastric pH was only affected by the omeprazole treatment, where it was predictably increased. Oxyntic contact angle was reduced by H. felis infection and this was partially reversed by amoxicillin or amoxicillin plus lactoferrin treatment thereby demonstrating the efficacy of lactoferrin and lactoferrin in combination with amoxicillin in treating H. felis infections.

FIG. 4 demonstrates the increased stomach mass in H. felis infection reflected in the increased ratio of stomach/body weight that was reversed by 35% in the lactoferrin-treated group.

FIG. 5 demonstrates partial reversals in contact angle changes from H. felis infection in the lactoferrin-treated group.

FIG. 6 demonstrates partial reversals in gastric pH changes from H. felis infection in the lactoferrin-treated group, where lactoferrin treatment resulted in about 50% reversals. The pH change is particularly notable as it indicates a return of acid-secreting parietal cells to the mucosa, cells whose disappearance is linked to atrophic gastritis and gastric cancer in man.

FIG. 7 demonstrates the response over time of changes in stomach mass in control mice (uninfected), H. felis-infected mice receiving vehicle, and H. felis-infected mice receiving lactoferrin or triple therapy. It can be seen that stomach mass was significantly different in infected versus uninfected mice. Further, lactoferrin treatment reversed the infection-induced changes by about 40%.

FIG. 8 demonstrates the response, over time of changes in gastric contact angle in control mice (uninfected), H. felis-infected mice receiving vehicle, and H. felis-infected mice receiving lactoferrin or triple therapy. It can be seen that gastric contact angle was significantly different in infected versus uninfected mice. Further, lactoferrin treatment reversed the infection-induced changes by about 40%.

FIG. 9 demonstrates the inflammation score in control mice, H. felis-infected mice receiving vehicle, and H. felis-infected mice receiving lactoferrin or triple therapy. That is, H. felis infection produced a significant increase in the presence of inflammatory cells in the gastric mucosa, and this increase tended to decrease by lactoferrin treatment.

FIG. 10 demonstrates that H. felis infection produced cell changes including the loss of parietal and chief cells seen in untreated, infected mice (FIG. 10B), compared to controls (FIG. 10A). This was partially blocked by lactoferrin after 15 days of treatment (FIG. 10C).

FIG. 11 shows the results from the dose-response study with lactoferrin, and the test of lactoferrin with and without amoxicillin. A discernible and significant increase in stomach mass was recorded in response to infection. These changes were reversed in a dose-dependent fashion when lactoferrin was intragastrically administered at 100 and 200 mg/kg/day.

FIG. 12 show the results from the dose-response study with lactoferrin, and the test of lactoferrin with and without amoxicillin. A discernible and significant decrease in gastric contact angle was recorded in response to infection. These changes were reversed in a dose-dependent fashion when lactoferrin was intragastrically administered at 100 and 200 mg/kg/day. The combination of lactoferrin and amoxicillin revealed a complete reversal of contact angle changes and a possible enhancement of stomach mass in infected mice. The effects of lactoferrin and amoxicillin on contact angle suggest that this combination is beneficial to the gastric mucosa in infected individuals.

FIG. 13 illustrates the response over time of changes in stomach mass for control (uninfected), H. felis-infected receiving vehicle, and H. felis-infected treated with rLF. There is a significant difference between the control (uninfected) and the H. felis-infected mice that received vehicle. The rLF treatment of infected mice resulted in a reduction of about 40% of that difference. These results demonstrate that orally administered rLF has the ability to reduce the progressive damaging effects of H. felis on the mouse gastric mucosa.

FIG. 14 illustrates the response over time of changes in gastric contact angle for control (uninfected), H. felis-infected receiving vehicle, and H. felis-infected treated with rLF. There is a significant difference between the control (uninfected) and the H. felis-infected mice that received vehicle. The rLF treatment of infected mice resulted in a reduction of about 40% of that difference. These results demonstrate that orally administered rLF has the ability to reduce the progressive damaging effects of H. felis on the mouse gastric mucosa.

FIG. 15 illustrates the response over time of changes in gastric pH for control (uninfected), H. felis-infected receiving vehicle, and H. felis-infected treated with rLF. There is a significant difference between the control (uninfected) and the H. felis-infected mice that received vehicle. The rLF treatment of infected mice resulted in a reduction of about 40% of that difference. These results demonstrate that orally administered rLF has the ability to reduce the progressive damaging effects of H. felis on the mouse gastric mucosa.

FIG. 16 shows the results of omeprazole treatment alone and omeprazole plus rLF on stomach mass. Omeprazole alone gave variable responses. Compared to H. felis infection (+vehicle), omeprazole treatment somewhat enhanced the gastric hypertrophy. In contrast, administration of rLF with omeprazole resulted in changes identical to rLF alone. FIG. 16 also shows that the triple antibiotic therapy was effective at reversing the changes that were induced by the bacteria.

FIG. 17 shows the results of omeprazole treatment alone and omeprazole plus rLF on gastric contact angles. Omeprazole alone gave variable responses. Compared to H. felis infection (+vehicle), omeprazole treatment somewhat reduced the reduction in contact angle. In contrast, administration of rLF with omeprazole resulted in changes identical to rLF alone. FIG. 17 also shows that the triple antibiotic therapy was effective at reversing the changes that were induced by the bacteria.

FIG. 18 shows the results of omeprazole treatment alone and omeprazole plus rLF on gastric pH. Omeprazole alone gave variable responses. Compared to H. felis infection (+vehicle), omeprazole treatment had no effect on pH. In contrast, administration of rLF with omeprazole resulted in changes identical to rLF alone. FIG. 18 also shows that the triple antibiotic therapy was effective at reversing the changes that were induced by the bacteria.

### SUMMARY OF THE INVENTION

H. pylori is an etiologic agent of gastric and peptic diseases. The organisms are invariably present when there is antral inflammation and successful treatment of H. pylori leads to resolution and healing of the gastric disease.

The present invention relates generally to the use of iron-binding glycoproteins and related polypeptides, and more specifically to lactoferrins and lactoferrin polypeptide fragments, in the treatment and prevention of disorders and diseases, microbial infections and viral diseases, including disorders and diseases related to H. pylori infection. More specifically, the present invention relates to the use of lactoferrin, or lactoferrin polypeptide fragments, in the prophylactic prevention of or the therapeutic treatment of enteropathogens such as E. coli, Salmonella spp., Shigella spp., H. pylori and Helicobacter spp. infections, either as a monotherapy or in combinations with antibiotics, antisecretory drugs, acid pump inhibitors, or H2-receptor antagonists, such as, but not limited to, tetracycline, bismuth subsalicylate or other bismuth compounds, metronidazole, amoxicillin, clarithromycin, omeprazole, Zantac, Tagamet, prescription or over-the-counter antacids, or other drugs. Preferably, the lactoferin is used in combination with one or more antibiotics. Lactoferrin or lactoferrin polypeptide fragments may also be used as a therapeutic or prophylactic treatment in combination with other immune modulators such as cytokines or free fatty acids.

In the preferred embodiment of the present invention, lactoferrin is administered orally. Used alone, or in combination with antimicrobials, or other therapeutics, lactoferrin may act as a bacteriostatic agent, sequestering iron and depriving bacteria of an essential nutrient, as a bactericidal agent by binding to microbes and promoting membrane disruption and lysis, and/or as an anti-attachment factor by binding to receptors on intestinal mucosa and preventing bacterial attachment and/or as an immune stimulant by binding to immune responsive cells to stimulate and promote immune response to invading microbes.

In particularly preferred embodiments, the lactoferrin is administered in an amount of at least 10 mg/kg/day, more preferably at least about 100 mg/kg/day, and in some embodiments about 200 mg/kg/day or even 400 mg/kg/day. Such doses may easily be modified without undue experimentation. In the most preferred embodiments, lactoferrin is administered in conjunction with one or more antibiotics such as, for example, amoxicillin.

Definitions.

For the purpose of the subject application, the following terms are defined for a better understanding of the invention.

The term “transferrin family” means a family of iron binding proteins including serum transferrin, ovotransferrin and lactoferrin. These proteins are structurally related.

The term “lactoferrin” means a protein molecule which is a member of the transferrin family.

The term “domain” is used to define a functional fragment of the lactoferrin protein or lactoferrin polypeptide which includes all or part of the molecular elements which effect a specified function such as iron binding, bactericidal properties, receptor binding, immune stimulation, etc.

The term “polypeptide” or “polypeptides” means several amino acids attached together to form a small peptide or polypeptide.

The term “substitution analog” or “allelic variation” or “allelic variant” each refer to a DNA sequence in which one or more codons specifying one or more amino acids of lactoferrin or a lactoferrin polypeptide are replaced with a different DNA sequence containing alternate codons that specify the same amino acid sequence. Where “substitution analog” or “allelic variant” refers to a protein or polypeptide, it means the substitution of a small number, generally five or less amino acids, as are known to occur in allelic variation in human and other mammalian proteins wherein the biological activity of the protein is maintained. Amino acid substitutions have been reported in the sequences of several published human lactoferrin proteins translated from cDNA sequences and these substitutions are most likely due to allelic variations.

The term “iron binding capacity” means ability to bind iron. Fully functional human lactoferrin can bind two atoms of iron per molecule of lactoferrin.

The term “biological activity” or “biologically active” means functional activity of lactoferrin as measured by its ability to bind iron, or kill microorganisms, or retard the growth of microorganisms, or to function as an iron transfer protein, or bind to specific receptors, stimulate immune response or regulate myelopoiesis.

### DETAILED DESCRIPTION OF THE INVENTION

### The Antibacterial Activity of Lactoferrin and its Polypeptide Fragments

Lactoferrin Therapy

The present invention is directed to the use of lactoferrin or lactoferrin polypeptide fragments, alone or in combination with compounds such as antibiotics, monoglycerides and/or free fatty acids, as a prophylactic or therapeutic treatment for disorders and diseases related to infection by enteropathogens such as H. pylori. In preferred embodiments, the lactoferrin or lactoferrin polypeptide fragments are used in combination with one or more antibiotics such as for example, amoxicillin. The present invention is further directed to pharmaceutically acceptable compositions thereof.

Combination Therapy: Lactoferrin and Antibiotics

Antibiotics, either singly or in combination, when combined with lactoferrin are highly efficient, more patient friendly and are a less expensive method of treatment for diseases related to H. pylori infection.

Antibiotics which may be used in the combination lactoferrin therapy include those antibiotics currently being used to treat H. pylori gastritis, including: (1) “Triple Therapy,” as described in Al-Assi et al., Am. J. Gastroent. 89:1203-1205 (1994), comprising administration of tetracycline HCl (or alternatively amoxicillin) (500 mg 4 times daily), bismuth subsalicylate tablets (2 Tablets, 4 times daily) and metronidazole (250 mg, 3 times daily) or alternatively, clarithromycin (500 mg, q.i.d.); and (2) “Double Therapy,” as described in Al-Assi et al., supra, (1994), comprising administration of amoxicillin (750 mg, 3 times daily) and metronidazole (500 mg, 3 times daily), or alternatively clarithromycin (500 mg, q.i.d.). For each of these treatment regimens, drugs are given for 14 days with meals, or with meals and at bedtime. In addition to the antibiotics and treatment regimens described above, other antibiotics, known today or developed in the future, may also be used.

Lactoferrin may be combined with each of the above antibiotics, anti-secretory drugs, acid pump inhibitors or H₂-receptor antagonists uniquely and in assorted combinations, varying both the dose and dosing frequency, to evaluate the synergy of lactoferrin combined with these drugs. The urea breath test, described by Graham et al., 1991, Am. J. Gastroent 86:1118-1122, may be used to monitor response to treatment. Specifically, response is considered positive if a significant reduction in the urea breath test results within 24 hours (of at least 20% reductions).

Effective lactoferrin combination therapy regimens which are related to the concurrent administration of antibiotics may be identified by studying healthy volunteers older than 18 years of age with active H. pylori infection, as shown by a positive serum antibody (IgG) to H. pylori and a positive ¹³C-urea breath test. Graham et al., Am. J. Gastroent., 1991, 86:1158-1162.

The urea breath test measures the magnitude of global urease activity in the gastrointestinal tract. Graham et al., Am. J. Gastroent., 1991, 86:1118-1122. Orally administered urea, labeled with either the stable isotope ¹³C, or the radioactive isotope ¹⁴C, is hydrolyzed by urease into ammonia and labeled carbon dioxide gas. The labeled gas appears in the expired breath following consumption, and when quantified, provides a straightforward, non-invasive method by which to measure the enzymatic urease activity of the gut. Gastrointestinal urease has been shown to be of microbial and not mammalian origin. And, because H. pylori is the most common urease positive gastric pathogen, an assay specificity and sensitivity of approximately 80% has been obtained for using the urea breath test. The test has provided a convenient measure of the gastrointestinal burden of urease positive microbes and for the evaluation of the effectiveness of prospective therapies used against them. Urea, prepared with the stable isotope ¹³C is the preferred substrate: it is available at low cost, may be administered repeatedly throughout life without contributing to the radioactive body burden, and is more ethically appropriate for children and pregnant women.

Patients receiving lactoferrin therapy or lactoferrin combination therapy regimens should receive a complete physical examination, routine blood and urine analysis, and urea breath test within 7 days of starting the study. A preferred study protocol would include approximately five oral administrations of lactoferrin in a 24 hour period over a duration of at least 7 and preferably at least 14 days. Dosage may be varied but typically will fall within a range of less than 5 grams total in any 24 hour period. Particularly preferred doses include at least about 10 mg/kg/day, at least about 100 mg/kg/day, at least about 200 mg/kg/day and even 400 mg/kg/day. Dosage may be optimized without undue experimentation to achieve maximum efficacy as skilled artisans will readily understand. Antibiotics will also be administered at a dose and dosing frequency consistent with previously established guidelines over the same 24 hour period.

At time 0, an initial EDTA blood sample (20 ml) may be drawn and the initial urea breath test administered to establish a baseline. At time 28 hours (4 hours after administration of the last lactoferrin dose), a second EDTA blood sample (20 ml) will be collected, and, a second urea breath test will be administered. A positive result will be defined as a ≧20% reduction in the urea breath test observed within 28 hours. Combination therapies having a positive response may be used to treat patients having diseases or disorders related to infection by enteropathogens such as H. pylori. It is also anticipated that the effective antibiotic dose and dosing frequency will be dramatically reduced when combined with lactoferrin for the treatment of diseases related to infection by enteropathogens such as H. pylori. It is anticipated that effective treatment and full compliance at reduced dosages will retard and delay development of antibiotics resistance for any given patient.

Combination Therapy: Lactoferrin and Monoglycerides an/or Free Fatty Acids

Combinations of lactoferrin and free fatty acids, in concentration ratios observed in human milk should be toxic to H. pylori and therefore promote recovery from H. pylori associated gastric disease and/or other disorders related to H. pylori infection as well as diseases caused by other enteropathogens such as E. coli, Salmonella and Shigella species.

The role of monoglycerides and free fatty acids as anti-infective agents in the neonate has been reviewed previously. Hamosh, “Free fatty acids and monoglycerides: Anti-infective agents produced during the digestion of milk fat by the newborn, “Immunology of Milk and the Neonate,” Advances in Experimental Medicine and Biology, Mestecky, J, et al., (Eds.), 1990, 310:151-158; Isaacs et al., (1990) Advances in Experimental Medicine and Biology, 130:159-165. Human milk typically has a fat content of 3-4% with free fatty acids released through action of lingual and gastric lipase. The free fatty acids with highest in vitro anti-infective activity are lauric (C12) and linoleic (C18:2) acids. It is believed that free fatty acids act through disruption of the surface membranes of bacteria and viruses. Further, it has been shown that H. pylori is sensitive to the toxic effects of unsaturated free fatty acids, the C20:4 arachidonic acid (CH₃(CH₂)₄(CH:CHCH₂)₄(CH₂)₂COOH; 5,8,11,14-eicosatetraenoic acid in particular. Hazell, J. Clin. Microbial., 1990, 28:1060-1061.

The concentration of various fatty acids found in human milk is shown in Table I as adapted from Casey and Hambidge, 1983, pp. 204, In: Lactation: Physiology Nutrition and Breast Feeding, Neville & Neifert (Eds).

TABLE I
Concentration of Fatty Acids in Human Milk

| | Colostrum (1-5 days) | | Mature Milk (>30 days) | |
|---|---|---|---|---|
| Total Fat (g/100 ml) | 2.9 | | 4.2 | |
| Fatty Acid | % Total Fat | g/100 ml | % Total Fat | g/100 ml |
| 12:0 Lauric | 1.8 | 0.0522 | 5.8 | 0.2436 |
| 14:0 Myristic | 3.8 | 0.1102 | 8.6 | 0.3612 |
| 16:0 Palmitic | 26.2 | 0.7598 | 21.0 | 0.8820 |
| 18:0 Stearic | 8.8 | 0.2552 | 8.0 | 0.3360 |
| 18:1 Oleic | 36.6 | 1.0614 | 35.5 | 1.4910 |
| 18:2 n-6 Linoleic | 6.8 | 0.1972 | 7.2 | 0.3024 |
| 18:3 n-3 Linolenic | 0 | — | 1.0 | 0.0420 |
| C₂₀ and C₂₂ | 10.2 | 0.2958 | 2.9 | 0.1218 |
| Polyunsaturated | | | | |

Lactoferrin may be combined with one or more of the above fatty acids, varying both the dose and dosing frequency, to evaluate the synergy of lactoferrin and free fatty acids. The urea breath test, described above, will be used to monitor response to treatment. In order to identify effective combination therapies, a study will be conducted using healthy volunteers older than 18 years of age with active H. pylori infection, as shown by a positive serum antibody (IgG) to H. pylori and a positive ¹³C-urea breath test Graham et al., Am. J. Gastroent., 1991, 86:1158-1162. Patients may receive a complete physical examination, routine blood and urine analysis, and urea breath test within 7 days of starting the study. Recombinant human lactoferrin (rhLf) may be administered orally (e.g. 5 times throughout a 24 hour period at selected doses, typically totalling less than 20 grams per 24 hour period). Particularly preferred doses include at least about 10 mg/kg/day, at least about 100 mg/kg/day, at least about 200 mg/kg/day and even 400 mg/kg/day Fatty acids will be administered orally at the same time. At time 0, an initial EDTA blood sample (20 ml) will be drawn and an initial urea breath test administered to determine a baseline level. At time 28 hours (4 hours after administration of the last lactoferrin dose), a second EDTA blood sample (20 ml) will be collected, and, a second urea breath test will be administered. A positive result will be defined as a significant reduction in the urea breath test within 28 hours (≧20% reduction). The specific lactoferrin and fatty acid combination demonstrating a positive response will be used in lactoferrin combination therapy as either a prophylactic or therapeutic treatment against enteropathogen infection such as H. pylori and diseases and disorders related thereto.

Production of Lactoferrin and Polypeptide Fragments Thereof

The lactoferrin of the present invention may be obtained from any relevant process, including purification of product from natural sources, protein synthesis and recombinant production.

The preferred embodiment is directed to pharmaceutical compositions and methods wherein recombinantly manufactured lactoferrin is utilized. As disclosed in U.S. Pat. Nos. 5,571,691, 5,571,896, 5,571,697, 5,766,939, 5,849,881 and co-pending U.S. Ser. Nos. 08/456,108, 08/691,123 and 08/866,544, the disclosures of which are herein incorporated by reference, efficient and economical methods for producing recombinant lactoferrin or polypeptide fragments thereof have been developed recently. The cDNA sequence encoding human lactoferrin can be used to prepare recombinant human lactoferrin, thus making available a source of protein for therapeutic and nutritional applications. The confirmed cDNA nucleotide sequence can be used in an appropriate cloning vehicle to replicate the cDNA sequence. Also, the cDNA can be incorporated into a vector system for human lactoferrin production. Other lactoferrin DNA sequences can be substituted for the human lactoferrin cDNA sequence to provide bovine, porcine, equine or lactoferrin from other species. Partial cDNA sequences can also be employed to produce desired lactoferrin polypeptide or polypeptide fragments. Such lactoferrin derived polypeptide or polypeptide fragments are not readily available by enzymatic digestion of naturally occurring lactoferrin. They are free of lactoperoxidase, lysozyme or other proteins that are contaminants of lactoferrin isolated from milk or other natural sources.

Lactoferrin may be produced in a variety of different organisms which permit integration of a vector comprising the lactoferrin cDNA or fragments thereof and the expression of such cDNA. Appropriate organisms include various fungi, such as Saccharomyces cerevisiae, Aspergillus nidulans, Apergillus oryzae, Aspergillus awamori, Kluyveromyces lectis and Pichia pastorsis, insect cells such as SF9, lactoferrin tolerant bacterial cells and mammalian cells such as Cos cells. The preferred host for expression of multigram quantities of recombinant lactoferrin is a eukaryotic cell. The preferred eukaryotic host cell is Aspergillus awamori. To maximize the antimicrobial activity of the recombinant lactoferrin of the present invention, in some embodiments of the invention, the lactoferrin may be acid treated prior to administration.

Pharmaceutical Formulations and Routes of Administration

In addition to the protocols set forth above, lactoferrin may also be administered to a patient, by itself, or in pharmaceutical compositions where it is mixed with suitable carriers or excipient(s) at doses to treat or ameliorate a variety of diseases and disorders related to microbial infections and viral diseases, including diseases related to H. pylori infection or to act in the prophylactic prevention of such diseases and disorders. A therapeutically effective dose further refers to the amount sufficient to result in amelioration of symptoms related to H. pylori infection or to prevent the onset of diseases or disorders related to H. pylori infection. Techniques for formulation and administration of the compounds of the instant application may be found in “Remington's Pharmaceutical Sciences,” Mack Publishing Co., Easton, Pa., latest edition.

Routes of Administration.

Suitable routes of administration may, for example, include oral, rectal, transmucosal, topical or intestinal administration; parenteral delivery, including intramuscular, subcutaneous, intramedullary injections, as well as intrathecal, direct intraventricular, intravenous, intraperitoneal, intranasal, or intraocular injections.

Alternately, the compound may be administered via a local route rather than a systemic manner, for example, in a depot or sustained release formulation. Furthermore, one may administer the drug in a targeted drug delivery system, for example, in a liposome containing lactoferrin and coated with an enteropathogen-specific such as an H. pylori-specific antibody. However, the most preferred route of administration is oral.

Composition/Formulation

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For the preferred oral administration, the compounds readily can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained as or from a solid excipient, optionally grinding a resulting mixture, and processing the mixture of granules, after adding suitable auxiliaries, if desired, to obtain tablets or draggee cores. Suitable excipients are, in particular but not limited to fillers such as sugars, including lactose, trahalose, sucrose, mannitol, or sorbitol; preparation such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Draggy cores are provided with suitable coatings. For this purpose, concentrated sugar solutions may be used, which may optionally contain gum arabic, talc, polyvinyl pyrrolidone, carbopol gel, polyehtylene glycol, and/or titanium dioxide, lacquer solutions, and suitable organic solvents or solvent mixtures. Dyesturrs or pigments may be added to the tablets or draggy coatings for identification or to differentiate various combinations of active compound doses.

Pharmaceutical preparations which can be used orally include push-fit capsules made of gelatin, as well as soft, sealed capsules made of gelatin and a plasticizer, such as blycerol or sorbitol. The push-fit capsules can contain the active ingredients in an admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such human administration.

Since H. pylori reside “outside” the body in the same way that gingival and skin bacteria do, a form of topical therapy is possible in the stomach. In the intragastric volume of the stomach varies from less than 50 ml during fasting to about 500 ml postprandially. Graham et al., 1990, Am. J. Gastroent., 1990, 85:1552-1555. If it is assumed that the intragastric volume is about 500 ml during the treatment phase i.e., with a meal, the administration of a bolus treatment can create local high concentrations in the gut.

For buccal administration, the compositions may take the form of tablets or lozenges formulated in the conventional and standard manner.

The compounds may also be formulated in rectal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include, but are not limited, to calcium carbonate, calcium phosphate, various sugars, starches, cellulose derivatives, gelatin, and polymers such as polyethylene glycols. The pharmaceutical compositions for each of the disclosed routes of administration may be formulated with or administered in combination with antibiotics, antisecretory drugs, acid pump inhibitors, H2-receptor antagonists, and other immune modulators.

Effective Dosage

Pharmaceutical compositions suitable for use in the present invention include compositions wherein the active ingredients are contained in an effective amount sufficient to achieve its intended purpose. More specifically, a therapeutically effective amount means an amount effective sufficient to prevent development of or to alleviate the existing symptoms of the subject being treated. Determination of the effective amounts is well within the capacity of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC50 as determined in cell culture. Such information can be used to more accurately determine effective and useful doses in humans.

A therapeutically effective does refers to that amount of the compound that results in the prevention of infection, amelioration of symptoms, or a prolongation of survival in a patient. Toxicity and therapeutic efficacy of such compounds can be determined by standard pharmaceutical procedures in cell cultures or experimental animals, e.g., for determining the LD50 (the does which is lethal to 50% of the population) and the ED50 (the dose therapeutically effective in 50% of the population). The dose ratio between toxic and therapeutic effects in the therapeutic index and it can be expressed as the ratio LD50/ED50. Compounds which exhibit large therapeutic indices are preferred. The data obtained from cell culture assays and animal studies can be used in formulating a range of dosage for use in human patients. The dosage of such compounds lies preferably within a range of circulating concentrations that include the ED50 dose and has little or no toxicity. The dosage may vary within this range depending upon the dosage form employed and the route of administration utilized. The exact formulation, route of administration and dosage can be chosen by the individual physician in view of the patient's individual clinical status and condition. (See, e.g., Fingle et al., 1975, in “The Pharmacological Basis of Therapeutics”, Ch. 1 p. 1). Dosage amount and treatment intervals also depend on and therefore are determined to produce micro-environmental levels of the active moiety sufficient to maintain the H. pylori inhibitory effects.

The amount of composition administered will, of course, be dependent on the patient being treated, on the patient's weight, the patient's mucosal surface area, the severity of the affliction, the manner of administration and the judgment of the prescribing physician. It has been determined that lactoferrin is effective at doses as low as about 10 mg/kg/day, at about 100 mg/kg/day, at about 200 mg/kg/day and at about 400 mg/kg/day. However, the dosage may easily be optimized without undue experimentation.

Packaging

The compositions may, if desired, be presented in a pack or dispenser device which may contain one or more unit dosage forms containing the active ingredient or other compounds to be administered in combination with the composition. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition. Suitable conditions indicated on the label may include treatment or prevention of infections related to infestation by bacteria, and more particularly, H. pylori.

### EXAMPLES OF THE PREFERRED EMBODIMENTS

The following examples are provided merely to exemplify the present invention and are not intended to be limiting. The invention is limited only by the scope of the appended claims.

### Example 1

Effect of In vitro Incubation of Recombinant Human Lactoferrin with H. pylori on its Subsequent Survival/Growth on Blood Agar Plates

A stock solution of recombinant human lactoferrin (rhLf) being <6% saturated with iron was prepared in 5 mM sodium phosphate buffer, pH 7.5, at a concentration of 21 mg/ml. Serial dilutions were then prepared to working concentrations of 3.0, 1.5, 0.75, 0.375 and 0.18 mg/ml. No particular attempt to exclude or limit iron-uptake by the protein was taken. Helicobacter pylori, collected and purified from 13 patients with duodenal ulcers were inoculated on blood agar plates containing brain, heart infusion (BMI) media supplemented with 7% horse blood. The plates were incubated at 37° C., 97% relative humidity and 12% CO₂ for 5 days. The number of organisms still able to grow following the incubation period was determined by counting the colonies. Table II summarizes the date from incubation for 48 hours at 1:10 dilution.

TABLE II
Survival/Growth of H. pylori Following 48 Hours Incubation
With Selected Concentrations of Recombinant Human Lactoferrin
In Vitro (Colony Forming Units at 1:10 Dilution)

| Mg/ml | 3.0 | 1.5 | 0.75 | 0.37 | 0.18 | PB |
|---|---|---|---|---|---|---|
| RhLf | | | | | | |
| Patient | | | | | | |
| #02 | 0 | 0 | 0 | 2 | 17 | tmc |
| #13 | 0 | 0 | | | 50 | tmc |
| #43 | 0 | 0 | 0 | 100 | | 100 |
| #22 | 0 | 0 | 0 | tmc | Tmc | tmc |
| #60 | 0 | 0 | 0 | tmc | Tmc | tmc |
| #21 | 0 | 0 | 14 | tmc | Tmc | tmc |
| #03 | 2 | 3 | 76 | tmc | Tmc | tmc |
| #40 | 14 | 3 | 31 | tmc | Tmc | tmc |
| #05 | 50 | 0 | 20 | tmc | Tmc | tmc |
| #38 | 120 | 120 | tmc | tmc | Tmc | tmc |
| #01 | 154 | 137 | tmc | tmc | Tmc | tmc |
| #18 | 200 | tmc | tmc | contam | Tmc | tmc |
| #55 | Tmc | tmc | tmc | tmc | Tmc | tmc |

Tmc = too many to count

The data demonstrated that recombinant human lactoferrin exhibited dose-dependent antibacterial activity against H. pylori in vitro when incubated under conditions of iron sufficiency. Growth was prevented in 6 of 13 patient isolates (patient nos. 02, 13, 43, 22, 60, and 21) following incubation with recombinant human lactoferrin at 3.0 mg/ml or 15 mg/ml for 48 hours. Growth was inhibited in varying amounts in an additional 6 of the 13 patient isolates and unaffected in an isolate from 1 patient at the highest concentration tested. Note also that 8 of 12 strains were killed or inhibited by 0. 75 mg/ml (about 10 μM). At 72 hours, there was no growth in 11 of 13 isolates (85%) at either 1.5 or 3.0 mg rhLf/ml. The results of this experiment are set forth in Table III.

TABLE III
Survival/Growth of H. pylori
Following 72 Hours Incubation With Selected Concentrations
of Recombinant Human Lactoferrin In Vitro
(Colony Forming Units at 1:10 Dilution)

| Mg/ml | 3.0 | 1.5 | 0.75 | 0.37 | 0.18 | PB |
|---|---|---|---|---|---|---|
| RhLf | | | | | | |
| Patient | | | | | | |
| #02 | 0 | 0 | 0 | tmc | tmc | tmc |
| #13 | 0 | 0 | | | 45 | tmc |
| #43 | 3* | 0 | 0 | tmc | tmc | tmc |
| #22 | 0 | 0 | 0 | tmc | tmc | tmc |
| #60 | 0 | 0 | 0 | tmc | tmc | tmc |
| #21 | 0 | 0 | 300 | tmc | tmc | tmc |
| #03 | 0 | 0 | contam | tmc | contam | tmc |
| #40 | 0 | 0 | 0 | tmc | tmc | tmc |
| #05 | 3* | 0 | 0 | tmc | tmc | tmc |
| #38 | 1* | 2* | tmc | tmc | tmc | tmc |
| #01 | 0 | 0 | tmc | tmc | tmc | tmc |
| #18 | TmC | tmc | tmc | contam | tmc | tmc |
| #55 | Tmc | tmc | tmc | tmc | tmc | tmc |

(wherein “tmc” = too many to count and “*” refers to “Not significant”).

Recombinant human lactoferrin appeared to be bactericidal rather than bacteriostatic against individual isolates of H. pylori, because, although incubation was performed in an iron limited media, the bacteria were returned to iron rich media of the blood agar plate for growth following the incubation. The return of a bacteriostatically suppressed organism to a nutrient sufficient media typically releases nutritional stasis and growth resumes.

### Example 2

Effect of Iron Deficient Recombinant, Iron Sufficient Recombinant, Iron Deficient Native and Iron Sufficient Native Human Lactoferrin on the In vitro Growth of H. pylori in an Iron Rich Environment of Blood Agar Plates

Different lactoferrin types and fragments will be tested against 20 clinical isolates of H. pylori. Recombinant human lactoferrin at ≦6% iron saturation and at 100% iron saturation, and, purified native human milk lactoferrin (Sigma) at <6% iron saturation and at 100% iron saturation will be incorporated into 7% blood agar plates as per the experimental design of Example 2. Concentrations of 0 (none), 0.75 mg/ml, 1.5 mg/ml and 3.0 mg/ml of each Fe-Lf will be incorporated into replicate blood agar plates. The plates will be incubated at 37° C., 97% relative humidity and 12% CO₂ for up to 5 days. The in vitro susceptibility of H. pylori for two sources of lactoferrin each presented at two extremes of iron saturation and three concentration inclusion levels will be evaluated by appearance of colonies on each plate. It is anticipated that there will be no difference in the bactericidal activity between either the source of the material or the iron saturation of each source, but rather activity will be a function of the inclusion concentration, irrespective of source or iron content.

### Example 3

Response of H. pylori Positive Patients to Treatment with Oral Recombinant Human Lactoferrin

Healthy volunteers older than 18 years of age with active H. pylori infection as shown by a positive serum antibody (IgG) to H. pylori and a positive C-urea breath test (Graham et al., Digest. Dis. and Sci, 1991, 36:1084-1088), will be studied. Patients will receive a complete physical examination, routine blood and urine analysis, and urea breath test within 7 days of starting the study. Recombinant human lactoferrin (rhlf) will be administered orally 5 times throughout a 24 hour period or longer, typically breakfast, lunch, dinner, bedtime and next breakfast at 1 gram per dose, for a total of 5 grams over the 24 hour study period. At time 0 (breakfast), an initial EDTA blood sample (20 ml) will be drawn and an initial urea breath test will be administered and rhlf (supplied in 1 gram packets as a dry powder) will be taken mixed in a small amount of liquid, typically about ¼ cup. The rhlf will be mixed in the water just prior to use, stirred until dissolved and consumed orally. At time 28 hours, a second EDTA blood sample (20 ml) will be collected, and, a second urea breath test will be administered. A positive result will be defined as a decrease in the urea breath test of >20%. The dose being administered to these adults is about 1/10th to 1/20th that consumed by a newborn infant, pound for pound. It is anticipated that the magnitude of the second urea breath test will be markedly less than the initial urea breath test, and that rhlf will be reducing the H. pylori population in vivo.

### Example 4

Effects of Enterally Administered Lactoferrin Against E. Coli

Study 1

Methods

Four-day-old Harlan Sprague-Dawley rats were given 1 cc of rh-lactoferrin at a dose of 1.0 mg/cc. The rh-lactoferrin was resuspended in 0.45% sterile normal saline for injection and appeared to completely dissolve. The rh-lactoferrin is not fully soluble in sterile water. The 0.45% saline was prepared from sterile water and sterile saline for injection 50-50 portions. Based on weights at the beginning and end of the study period, the dose of rh-lactoferrin ranged from about 500 to 1000 mg/kg with an average of 750 mg/kg/day for the duration of the study. The reconstituted rh-lactoferrin was delivered by passing a 3 Fr. silastic catheter (Gesco) to the stomach (approximately 3 to 4 cm) followed by slow injection. Measurement of the distance was determined via the method used to pass gavage tubes in human newborns. The control group, which subsequently was infected enterally with E. coli in parallel with the rh-lactoferrin treated animals, was not subjected to gavage with the vehicle 0-45% saline

Three liters of typticase soy agar (TSB) were inoculated with a single loop from a blood agar plate on which was grown an entero-invasive strain of E. coli. After overnight incubation, the bacteria were isolated by centrifugation (IEC refrigerated table top centrifuge at 3000 rpm for 15 min at 4° C.), washed once by centrifugation in sterile saline, and the pellets resuspended in 10 mL of sterile saline (final volume=approx. 25 mL). One-half ml of this slurry was instilled into the stomach of the two groups of rats (non-treated and rh-lactoferrin-treated litters) using a 3.5 Fr. polyurethane umbilical artery catheter (Argyle). Again, the catheter was passed to the stomach, a distance of 3 to 4 cm. No rats had died after 2 days of infection. For this reason, the bacterium was switched to E. coli strain EcS, and grown overnight and prepared for GI infection the following day. Strain Ec5 was then used for enteral infection by giving a dose of one mL of the slurry by gavage into the stomach of each pup from both groups. This bacterial slurry was at a concentration of about 4×10¹⁰ organisms per mL. The pups averaged 21 to 22 grams body weight at this point, so the effective infective dose was ≧2×10¹² E. coli per kg. The pups had a mean weight of 15 grams prior to innoculation. In 48 hours, all pups in the rh-lactoferrin group were alive and seemed active, pink and without distress. Two pups were missing from the non-treated, E. coli-infected group, one pup was dying (dyspnea, cyanosis and unresponsiveness), one pup appeared ill (lethargic and gray), and all the remaining 5 pups in this group seemed less ill, but still ill in appearance. The pups in both groups were sacrificed by intracisternal pentobarbital. Thereafter, the substernal area was cauterized, and a 20 gauge needle inserted and blood was aspirated from the heart (0.1 to 0.8 ml). This blood was immediately inoculated into 5 ml of trypticase soy broth. After overnight incubation at 37° C., the broth was subcultured onto 5% sheep blood agar. Within 24 hours, there was positive growth of E. coli on the blood plates for each animal in both groups. The mean weights in the two groups varied by about one gram with the non-treated, infected group being the less of the two weights.

Results

Observation revealed that in the non-treated, infected group 2 were missing, 1 was dying, 1 was ill, 5 were surviving, and 7 had positive blood cultures. Thus, there were 4 dying, ill or missing rat pups from the total of 9. Observation revealed that in the lactoferrin-treated, infected group there were no missing, dying or ill pups with 9 surviving and all 9 having positive blood cultures. Thus, there were 0 dead, dying, ill or missing rat pups of from the total of 9.

Study 2

Methods

The experiment was repeated. Four-day-old Harlan Sprague-Dawley rats arrived in two litters of 10 pups each. The dams were proven breeders. On the consecutive days, the rats received 1 cc of rh-lactoferrin at a dose of 10 mg/cc. The rh-lactoferrin was resuspended in 0.45% sterile normal saline for injection and appeared to completely dissolve. The 0.45% saline was prepared from sterile water and sterile saline for injection (50-50 proportions). Based on weights at the beginning and end of the study period, the dose of rh-lactoferrin ranged from about 500 to 1000 mg/kg with an average of 750 mg/kg/day. The reconstituted rh-lactoferrin was delivered by passing a 3 Fr. silastic catheter (Gesco) to the stomach (approximately 3 to 4 cm) followed by slow injection. Measurement of the distance was determined as in Study 1.

Three liters of trypticase soy agar (TSB) were inoculated with a single loop from a blood agar plate on which was grown an enteroinvasive strain of E. coli (strain Ec5). After overnight incubation at 37° C. with shaking, the bacteria were isolated by centrifugation (IEC refrigerated table top centrifuge at 3000 rpm for 15 min at 4° C.) and the pellets resuspended in 10 ml of sterile saline (final volume=approx. 25 ml). In about 24 hours, one-half ml of this slurry was instilled into the stomach of the two groups of rats (non-treated and rh-lactoferrin-treated litters) using a 3.5 Fr. polyurethane umbilical artery catheter (Argyle). Again, the catheter was passed to the stomach, a distance of 3 to 4 cm. The E. coli slurry was at a concentration of about 4×10¹⁰ organisms per ml. The pups averaged 15 grams body weight at this point, so the effective infective dose was ≧1-2×10¹² E. coli per kg.

Twenty-four hours after the initial enteral infection, three animals in the non-lactoferrin, E. coli-infected group were dead or dying. One pup in the lactoferrin-treated, E. coli-infected had died or was dying while the remaining pups appeared active, pink and well. In about 24 hours, a second instillation of E. coli was performed. The dying pups were dyspneic, gray, and unresponsiveness with bicycling behavior of the legs. These dying pups were sacrificed by intracisternal pentobarbital. Thereafter, the substernal area was cauterized, and a 20 gauge needle inserted and blood was aspirated from the heart (0.1 to 0.5 ml). The blood (0.1 ml) was immediately inoculated onto a 5% sheep blood agar plate and streaked with a sterile loop. An additional 0.1 ml was mixed with 0.9 ml of sterile water to lyse the WBCs (and RSCS) and release ingested bacteria. The 10-1 blood lysate was serially diluted in sterile saline, and 0.1 ml was dripped onto 5% sheep blood agar and spread with a sterile loop. After 24 hours of incubation at 37° C., the colonies were counted with a plate counter. Additionally, the abdomen was aseptically opened, and the bowel and liver was inspected. Thereafter a section of liver was obtained and a touch preparation made on 5% sheep blood agar. The dead rat pup also had blood obtained from the heart which was plated on 5% sheep blood agar and a touch-prep of the liver on blood agar was also performed.

In about 24 hours, the second 1 ml of a freshly-prepared E. coli slurry was instilled by gavage into the stomachs of the surviving rat pups of both groups. Twenty-four hours after the second enteral infection, two animals in the non-lactoferrin, E. coli-infected group were dead, and the remaining animals appeared ill to variable degrees while all pups in the lactoferrin-treated, E. coli-infected group appeared active, pink and well. The mean weights in the two groups were determined. The two dead rats and all the surviving pups of both groups underwent study as described above. This included CFU determinations of blood aspirates from the hearts and touch cultures of the livers.

Results

Observation revealed that in the non-treated, E. coli infected group 3 were dead and 2 were ill and dying rat pups of 10 total. All pups in this group, whether dead, dying or surviving had positive blood and liver cultures. The blood cultures, on average, were a lot higher in the surviving pups compared to those cultured from the lactoferrin-treated, E. coli infected group. The liver touch cultures also had higher CFU than did those same cultures in the lactoferrin-treated, infected group.

Obervation revealed that in the lactoferrin-treated, E. coli infected group one was missing and 0 dead, dying, or ill rat pups of 10 total. Of the 9 surviving rat pups, 8 had positive blood cultures and 9 had positive liver cultures. The number of CFU in blood and liver were, however, much less in the lactoferrin-treated, infected group versus the nontreated, infected group. For example, the CFU in blood was a lot lower, on average, in the lactoferrin-treated, infected group versus the non-treated, infected group. The CFU of the liver touch preparations were also less in the lactoferrin-treated, E. coli infected group versus the non-treated, infected group.

### Example 5

Human Lactoferrin Impairment of Shigella flexneri Virulence

The relevance of lactoferrin in protection from invasive enteropathogens including Shigella spp., Salmonella spp., and enteroinvasive E. coli has not previously been defined. Breastfeeding decreases the severity of Shigella spp. infection in infants who become colonized early in life. (Guerrero et al., Pediatr Infect. Dis. J. 13:597 (1994)).

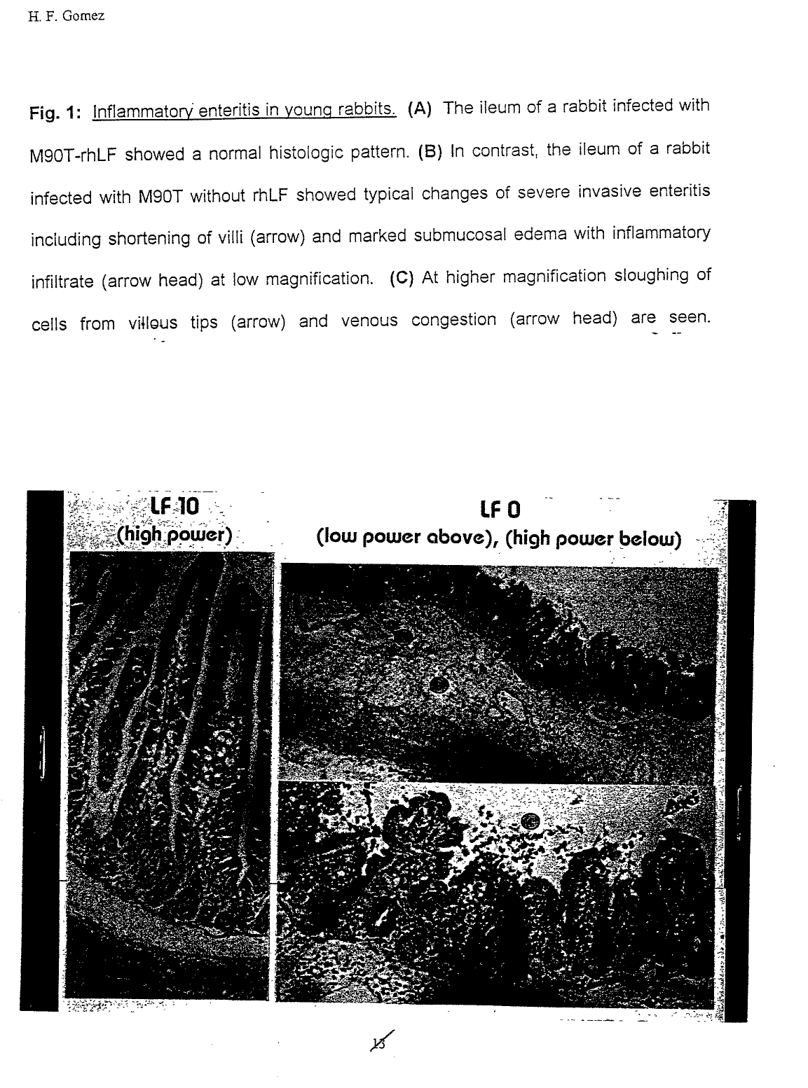

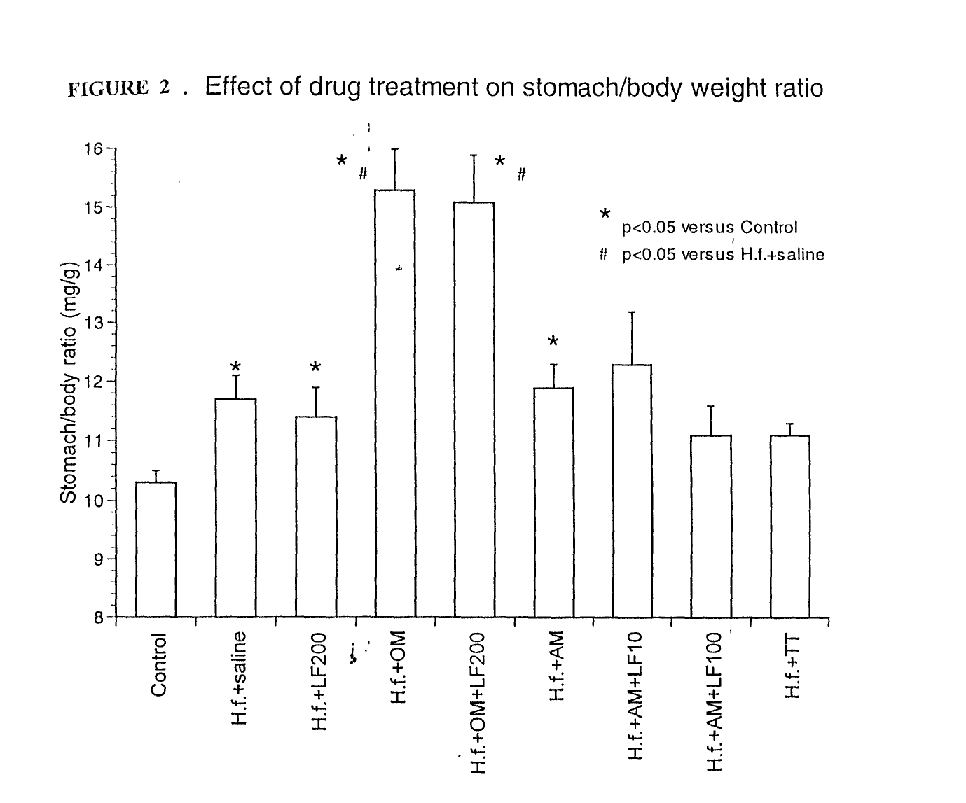

The invasiveness in vivo and in vitro of rhLF-treated Shigella flexneri 5 strain M90T (M90T-rhLF) was compared to that of control M90T not treated with rhLF (M90T). Inflammatory enteritis developed significantly more often in 4 week old New Zealand White rabbits infected with M90T than in those infected with M90T-rhLF (22/34[65%] vs 4/34[12%], p<0.001). Typical histologic changes included villous blunting with sloughing of epithelial cells, submucosal edema, infiltration of leukocytes, venous congestion, and hemorrhage of ill animals sacrificed 24 hours after infection as depicted in FIG. 1. The degree of inflammation was significantly greater in the M90T than in M90T-rhLF treated animals.

This decrease in inflammation suggested that the ability of S. flexneri to invade was impaired. Therefore, to determine whether rhLF might be impairing virulence, organisms were briefly exposed to rhLF and their ability to invade mammalian cells determined. A one hour pre-incubation with rhLF (10 mg/mL) impaired the ability of S. flexneri strain M90T to invade HeLa cells as indicated by a decrease in CFU in a gentamicin overlay assay-, the CFU done in triplicate four times of M90T was 7.3±0.7×10⁶ compared to 2.7±0.5×10⁶ for M90T-rhLF (p<0.001). A decrease in CFU after exposure to rhLF could reflect impaired adherence, disordered internalization or sluggish multiplication of bacteria. Bacterial adherence was assessed using ³H-labeled S. flexneri briefly incubated with HeLa cells, washed, and lysed by detergent-. Adherence was not affected by rhLF (13±10 M90T/HeLa cell versus 13±8 M90T-rhLF/HeLa cell triplicate assays done three times). Growth of M90T-rhLF and M90T was determined. A 60 minute exposure to rhLF was not bacteriostatic. The generation time in brain heart infusion broth was 104 minutes compared to 106 minutes for M90T and M90T-rhLF, respectively (assays done in triplicate twice). Thus, the internalization process became the major focus of these studies.

Internalization of Shigella species is encoded by a set of genes located primarily on a 230-kb plasmid. Within this large plasmid there is a 31 kb region that encodes for the invasion plasmid antigen genes (ipa), as well as genes for their membrane expression (mxi) and surface presentation (spa). The ipa genes encode proteins (Ipaa, Ipab, Ipac, and Ipad) that are essential to the invasion phenotype and are the dominant antigens in the humoral response to Shigella species infection. Ipab, Ipac, and Ipad are essential for invasion while the role of Ipaa is less well established. The initial steps in uptake of Shigella species are under the control of the Ipabc complex. Ipab and Ipac are transported to and expressed on the bacterial cell surface where they become associated as a complex and are secreted by the Mxi-Spa type III secretary mechanism. Ipab and Ipac are unstable unless complexed intracellularly with a molecular chaperone, Ipgc, or extracellularly with each other. This complex alone is adequate to trigger uptake-latex particles coated with Ipabc are rapidly taken up by HeLa cells and induce actin polymerization. The effect of exposure of S. flexneri to rhLF on the formation and stability of the Ipabc complex was therefore determined. The supernatant of M90T-rhLF was examined by immunoblot for presence of invasion plasmid antigens using both polygonal and monoclonal antibodies. Immunoblots reacted with polyclonal convalescent human anti-serum showed that lpab was rapidly lost from the bacteria in an unprotected and unstable form. The instability suggested that lpab and lpac were not associated with Ipgc or with each other. The amount of invasion plasmid antigens released and degraded related to the amount of rhLF with which the S. flexneri had been incubated. Loss of Ipabc was not related to the iron-binding properties of lactoferrin because saturation with ferric iron was not associated with a decrease in loss of invasion plasmid antigens or a change in the degradation pattern. To determine whether rhLF acted on the Ipabc complex, a cell-free invasion plasmid antigen preparation was incubated with various concentrations of rhLF. Dissociation and degradation of the Ipabc complex was not induced by rhLF. Therefore, rhLF likely acts at the cell surface to displace Ipab and Ipac rather than causing dissociation of the secreted IpaBC complex. Immunoblots reacted with monoclonal antibodies confirmed these findings and also showed that Ipad was not lost from the cell surface and only trace amounts of IcsA were lost after rhLF treatment. The amount of Ipab and Ipac released and degraded was related to the amount of rhLF to which the organism was exposed. SDS-PAGE demonstrated that the proteins released by rhLF were not the same as those found in a whole cell lysate. Thus, rhLF-mediated cell lysis was not occurring.

Loss of Ipab and Ipac in a non associated unstable form after exposure to rhLF is unique. Other stimulus (cell contact, exposure to serum or incubation with dyes) that cause release of these proteins induce the secretion of a stable Ipabc complex. Since Ipab and Ipac appear not to be synthesized during intracellular multiplication, loss of Ipab and Ipac prior to formation of the Ipabc complex could abort infection. There are several possible mechanisms for this specificity (release of lpab and lpac without release of other invasion plasmid antigens). rhLF could interact with an outer membrane Mxi-Spa protein involved in the assembly of the lpabc complex. Alternatively, rhLF could interact directly with both Ipab and Ipac but not other invasins at a point when Ipab and Ipac are in physical proximity on the cell surface. It is also possible that rhLF disrupts LPS-outer membrane protein interactions. There are two binding sites for LPS on lactoferrin: a high affinity site in the N terminal domain and a low affinity site in the C terminal domain. The cationic portion of lactoferrin may disrupt cell surface association of proteins with LPS and/or with each other. rhLF caused release of LPS as has been shown by others (Dalmastri et al., Microbiologica 11:225 (1988); Ellison 3d et al., Infect. Immun. 56:2774 (1988)). LPS is an essential virulence factor in Shigella species. Its role in virulence may be in organizing, orienting, and presenting the major virulence proteins.

The virulence mechanisms of Shigella spp. are similar to those of other enteropathogens. Enteroinvasive E. coli possess virulence genes virtually identical with those of Shigella species (Hsia et al., J. Bacteriol. 175:4817 (1993)) S. typhimurium, S. typhi, and S. dublin have chromosomally encoded invasion proteins homologous to Ipaa-d of Shigella species as well as genes closely related to the mxi/spa translocon of the type III protein secretion system. Thus, the mechanism by which lactoferrin impairs virulence in Shigella species may be mimicked by similar effects on other Gram negative enteric pathogens that express closely related outer membrane-anchored virulence proteins.

### Example 6

Effect of Lactoferrin, Amoxicillin, and Omeprazole on H. felis Infection in Mice

H. felis in mice serves as a model for studying H. pylori infection in humans. A urea breath test was used in the present experiment in order to detect Helicobacter infection and to assess the ability of test drugs to eradicate the infection in a simple, non-invasive manner. The basis of this test is that radiolabelled urea is administered orally to a mouse, and if the Helicobacter bacteria is present in the stomach, its high urease activity will split the urea to ammonia and labelled carbon dioxide. The carbon dioxide is then rapidly absorbed and expelled through the breath, which is collected in a carbon dioxide trap and counted. An animal with no bacteria/urease in the stomach will give very low counts, while an infected animal will yield a high number of counts.

Methods

C57BU6 female mice were infected with H. felis (ATCC 49179) and used after 4 weeks. For the treatment alms of this study, mice were administered orally each day a volume of 0.1 ml containing either saline (control), lactoferrin (200 mg/kg), amoxicillin (18-75 mg/kg), amoxicillin (18.75 mg/kg) plus lactoferrin (10 or 100 mg/kg), or triple therapy (metronidazole, tetracycline, bismuth subsalicylate). Other mice received intraperitoneally (ip) omeprazole (150 mg/kg) or omeprazole (150 mg/kg, ip) plus lactoferrin (200 mg/kg, orally). After two weeks of treatment, mice were euthanized and gastric tissues collected for analysis of stomach weight, pH, contact angle, histology, and urease (biopsy test).

For the urea breath test, mice were fasted overnight and then administered orally 0.2 ml of ¹⁴C-urea (0.5 μCi) in water. Each mouse was immediately placed in a sealed chamber through which air flowed at −80 cc/min and which then was bubbled into an alkaline trapping solution (4 m] of 150 mM benzethonium hydroxide). The outflow from the chamber was collected for 9 min, after which it was combined with scintillation fluid and counted for radioactivity.

Results

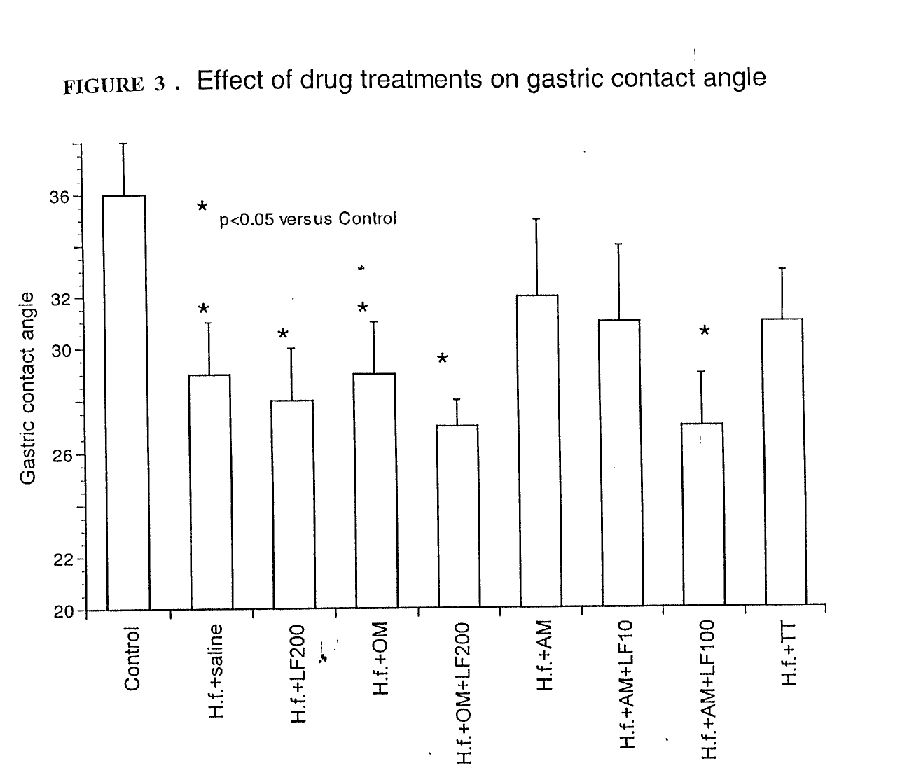

The data from the drug treatment study is summarized in Table IV. It was found that body weights did not differ between treatment groups. Stomach weight alone appeared to increase in the H. felis-infected animals, but it did not reach statistical significance. Stomach mass was, however, increased in omeprazole-treated infected mice, regardless of whether they received lactoferrin or not. The stomach/body weight ratio was significantly higher in infected mice and was reduced somewhat by lactoferrin alone and the combination of low-dose amoxicillin and lactoferrin, but not by omeprazole and lactoferrin (FIG. 2). Gastric pH was only affected by the omeprazole treatment, where it was predictably increased. Oxyntic contact angle was reduced by H. felis infection and this was partially reversed by amoxicillin or amoxicillin plus lactoferrin treatment (FIG. 3). Biopsy urease testing revealed no bacteria in uninfected controls, a nearly complete infection rate in saline-treated H. felis mice, and reduced infection rates in treated groups receiving lactoferrin or amoxicillin alone, and amoxicillin plus lactoferrin.

The urea breath test was applied to these animals the day before sacrifice and results are presented in Table V. Control uninfected animals, as well as infected animals, gave occasional high readings which created considerable variability and precluded statistical analysis. The source of this variability is probably related to the presence of fecal matter and its accompanying bacteria in the stomach of coprophagic animals.

Lactoferrin at 200 mg/kg/day reduced some of the manifestations of Helicobacter infection in mice. The combination of lactoferrin (200 mglkg) with omeprazole offered no advantage, while the combination of lactoferrin (100 mglkg) and amoxicillin gave beneficial effects.

Experiment 7 Effect of Lactoferrin, Amoxicillin, and Omeprazole on H. felis Infection in Mice Infected for More than 6 Months

Experiment 6 demonstrates that a relatively acute infection of H. felis in mice (about 4 to 6 weeks) can be treated with human lactoferrin. However, clinically most Helicobacter infections in man are of a chronic nature and may be more difficult to treat. Therefore, the present study was designed to test lactoferrin in mice that had been infected with H. felis for more than 6 months.

Methods

Five mice had been infected with H. felis. They were maintained in micro-isolator cages for approximately 7 months, when they were divided into treatment groups. Three mice were treated with lactoferrin (200 mg/kg) in saline and two mice were treated with the vehicle (saline) daily by mouth for 3 weeks. There were also a group of three uninfected, age-matched control mice. Following the last dosing, the mice were fasted overnight to ensure an empty stomach. They were euthanized and gastric tissue collected for analysis of stomach weight, contact angle, pH, and presence of bacteria (biopsy urease test).

Results

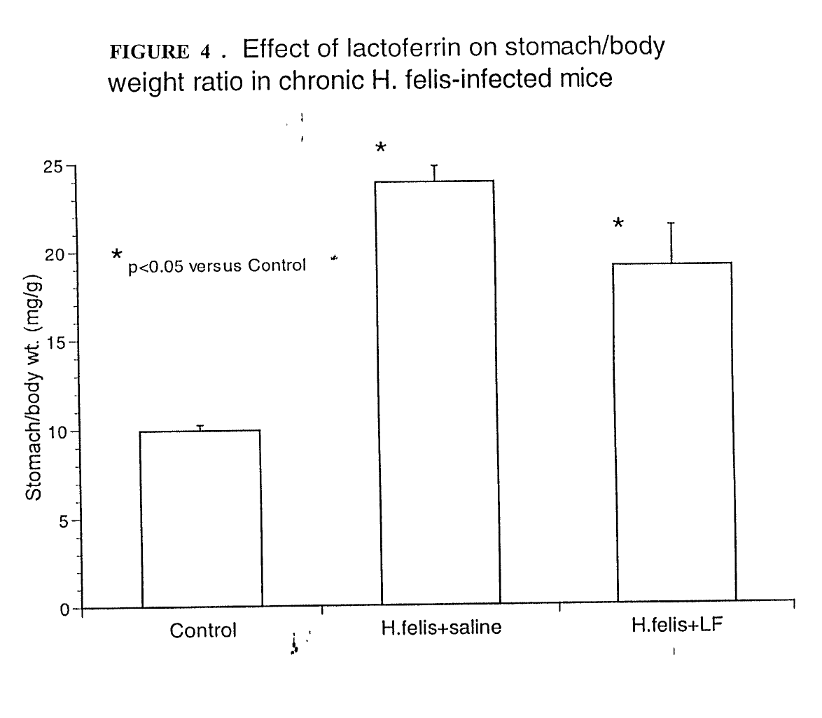

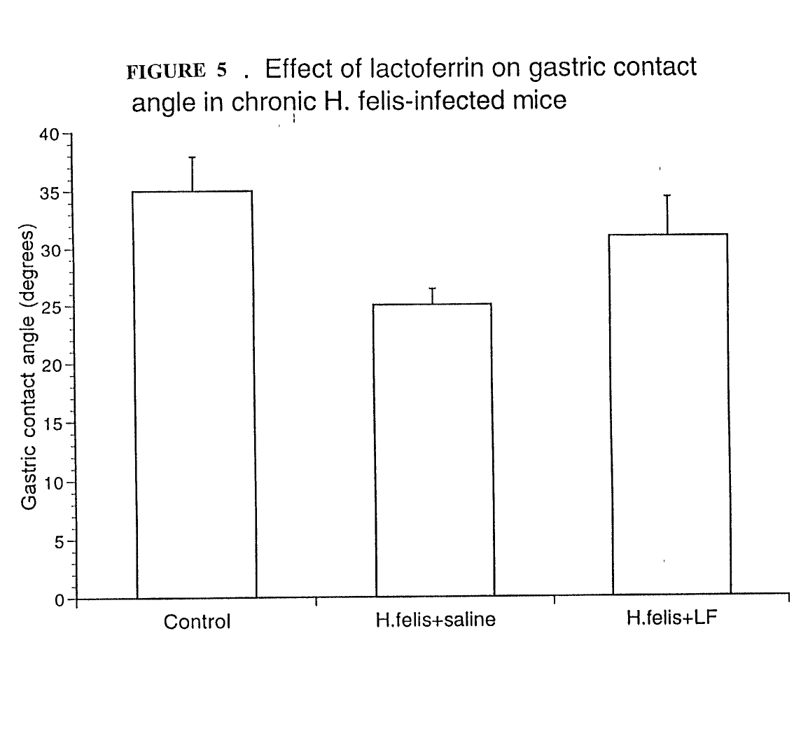

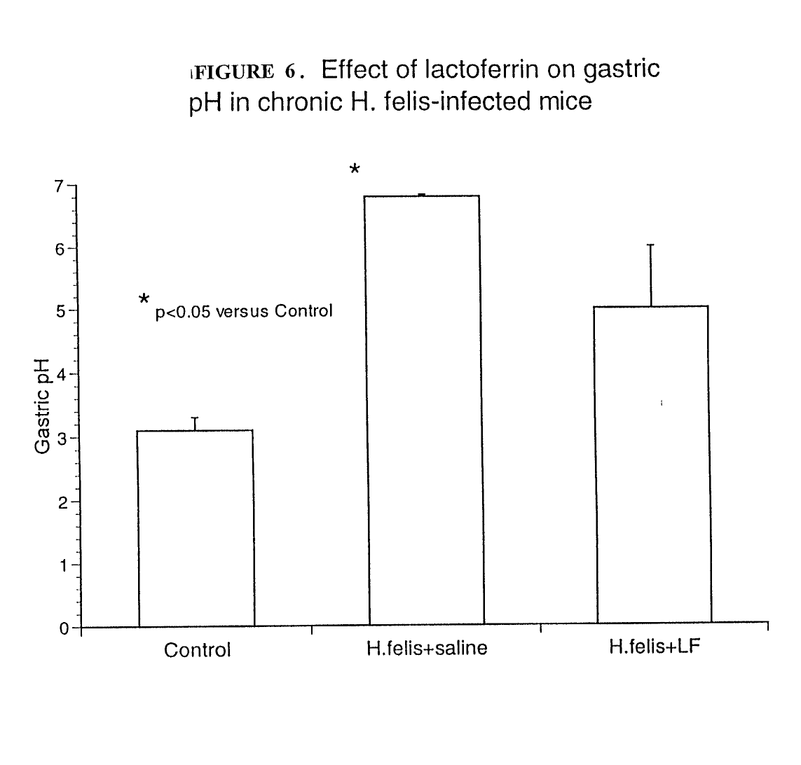

The data are summarized in Table VI. It was found that the body weights did not differ between the uninfected and either of the infected groups, but that there was a large increase in stomach mass in both the saline and lactoferrin-treated infected mice. This increased stomach mass was reflected in the increased ratio of stomach/body weight that was reversed by 35% in the lactoferrin-treated group (FIG. 4). Similar partial reversals were also seen in contact angle changes (FIG. 5) and gastric pH (FIG. 6), where lactoferrin treatment resulted in about 50% reversals. The pH change is particularly notable as it indicates a return of acid-secreting parietal cells to the mucosa, cells whose disappearance is linked to atrophic gastritis and gastric cancer in man. Urease testing failed to indicate the presence of bacteria in either the uninfected controls or the infected mice receiving saline. There was one out of three positive in the lactoferrin-treated group. The absence of bacteria in these animals is not unexpected, as the bacteria do not grow well in a neutral pH environment and are known to disappear from patients with atrophic gastritis, while the damage caused by the bacteria persist.

Lactoferrin treatment of chronic H. felis-infected mice was effective at partially reversing key manifestations associated with glandular atrophy. Lactoferrin actions on the stomach may include more than simple antibiotic activity. The finding that lactoferrin could initiate reversal of atrophic changes in the glandular regions of the stomach strongly suggests that it may be useful in treating such changes in man which are known to lead to gastric cancer. The use of lactoferrin in conjunction with an antibiotic such as amoxicillin hastens the reversal of Helicobacter induced changes and may prevent subsequent development of cancer.

Experiment 8 Efficacy of Human Lactoferrin in Combination with Amoxicillin in Treating H. felis Infection in Mice

Background

The aim of these studies was to determine the efficacy of recombinant human lactoferrin at treating a gastric infection of Helicobacter felis in mice, as a model for the use of lactoferrin in Helicobacter infections in man. Presently, the most well established animal model for gastric Helicobacter infection is that of the mouse infected with Helicobacter felis (Gastroenterology 99:1315, 1990). This infection is chronic and produces a progressive hypertrophy of the stomach with atrophy of parietal cells and other differentiated gastric epithelial cells (Gastroenterology 110: 155,1996).

The gastric hypertrophy in H. felis-infected mice consists of hyperplasia of foveolar mucous neck cells which appear to overgrow and displace the former acid, pepsin, and endocrine secretory cells. This hypertrophy is measurable by weighing the mass of the stomach and then normalizing it to the animal's body weight. Previous studies have shown little or no change in body weight upon infection. Therefore, determination of the stomach body weight ratio becomes a measure of infection.

Accompanying the infection is a loss of the integrity of the gastric mucosal barrier. Quantitation of the surface hydrophobicity of the mucosa may be accomplished by determination of the contact angle which forms when a micro-droplet of water is placed on the mucosal surface and the angle which forms at the triple point between the mucosa, air, and liquid is measured. This technique has been used to show that an intact mucosa has a relatively hydrophobic surface (high contact angle), but that a disruption of this surface, such as occurs with gastric damaging agents or Helicobacter infection, results in a reduction in the contact angle (Annu. Rev. Physiol. 57:565 (1995)). This measure becomes a parameter of infection.

A final measure of infection can be obtained from histological examination of hematoxylin and eosin-stained gastric tissue. This will confirm the state of inflammation of the stomach and its associated changes in cell types.

Methods

C57BU6 female mice were infected with viable cultures of Helicobacter felis (ATCC 49179) by oral inoculations of 0.2 ml per mouse (10¹⁰ colony forming units/ml) three times at two day intervals. Age-matched control (uninfected) mice were maintained in parallel. At predetermined time points, measurements were made of fasting body weight, stomach weight, contact angle of oxyntic tissue, and histological presence of parietal and chief cells and inflammation score. The inflammation score was determined by an observer who was unaware of the treatment. The score ranged from 0 (no inflammatory cells present) to 6 (heavy infiltration of inflammatory cells throughout the gastric mucosa and submucosa).

Study 1

The purpose of this study was to test a single dose of lactoferrin (100 mg/kg/day) alone and in combination with the proton-pump inhibitor omeprazole (150 mg/kg/day), for efficacy against H. felis infection. Two groups of H. felis-infected mice were treated daily by a single oral inoculation of lactoferrin. After 15 days of treatment, one group of mice was euthanized and samples were collected and analyzed. The additional group was treated with lactoferrin for 15 days and was left untreated for 15 days. At each of these time points, control (uninfected) mice and H. felis-infected mice that had received vehicle daily (0.1 ml water) were also assessed. In addition, three other groups of mice were treated daily for 15 days with either omeprazole, omeprazole plus lactoferrin, or triple therapy which is the standard treatment for Helicobacter infection (tetracycline, 1.5 mg; metronidazole, 0.675 mg; and bismuth subcitrate, 0.185 mg). After an additional 15 days without treatment, these latter groups were also assessed.

Study 2

The purpose of this study was to perform a dose-response with lactoferrin (100, 200, and 400 mg/kg/day) against H. felis infection, and to compare lactoferrin alone (100 mg/kg/day) and in combination with the antibiotic amoxicillin (37.5 mg/kg/day). Groups of H. felis-infected mice were treated daily by a single oral inoculation of lactoferrin alone, amoxicillin alone, lactoferrin plus amoxicillin, or triple therapy (tetracyline, metronidazole, and bismuth subcitrate). After 14 days of treatment, mice were euthanized and samples were collected and analyzed. At this time, control (uninfected) mice and H. felis-infected mice that had received vehicle daily (0.1 ml saline) were also assessed.

Results

Study 1.

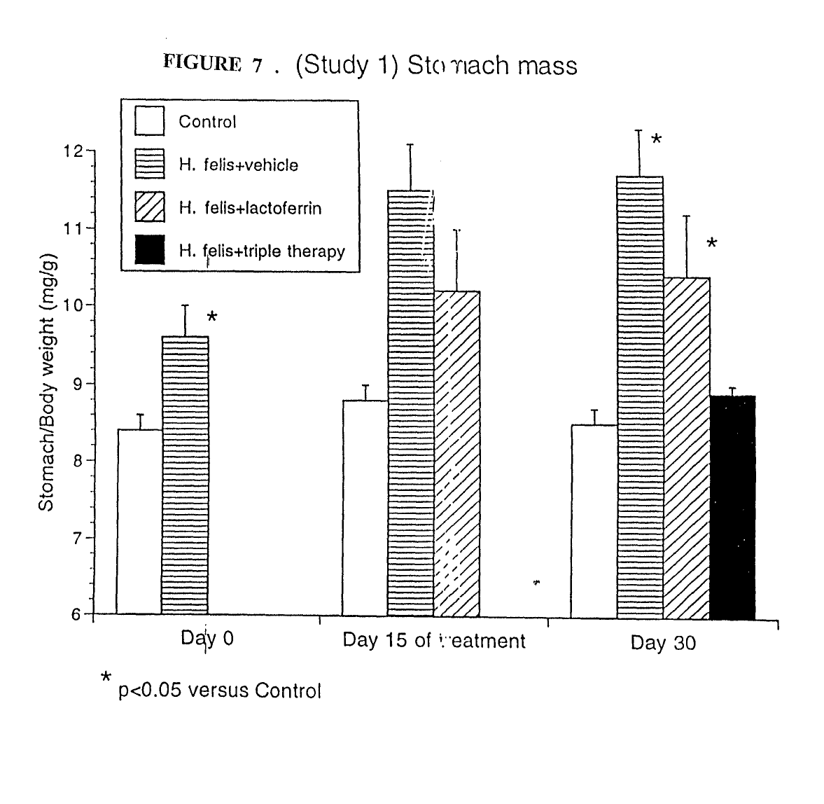

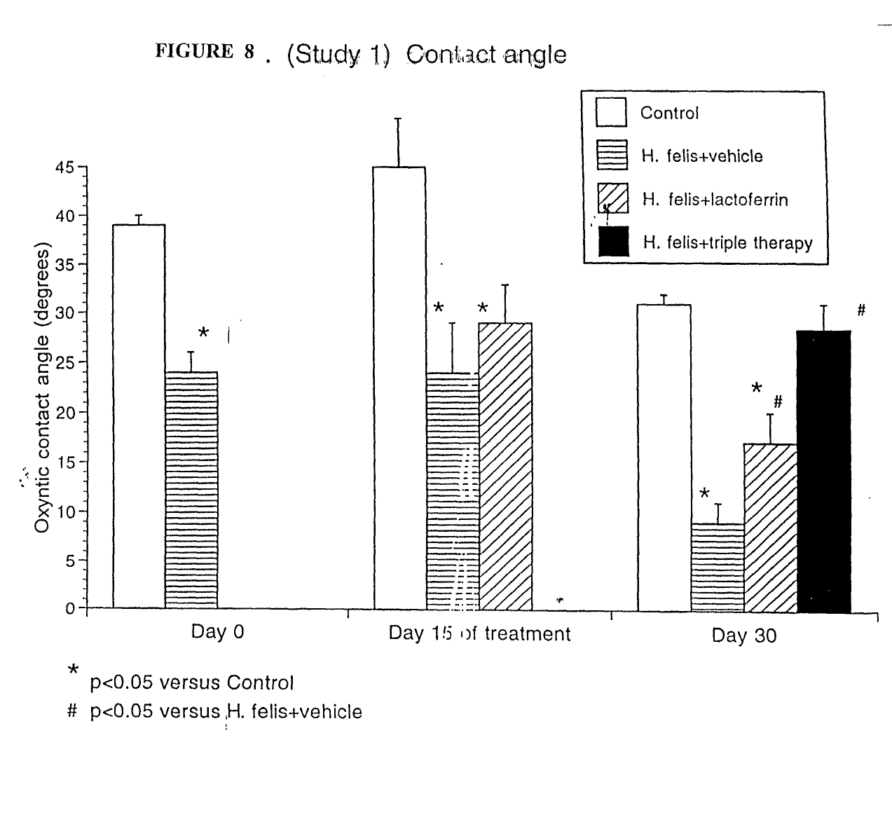

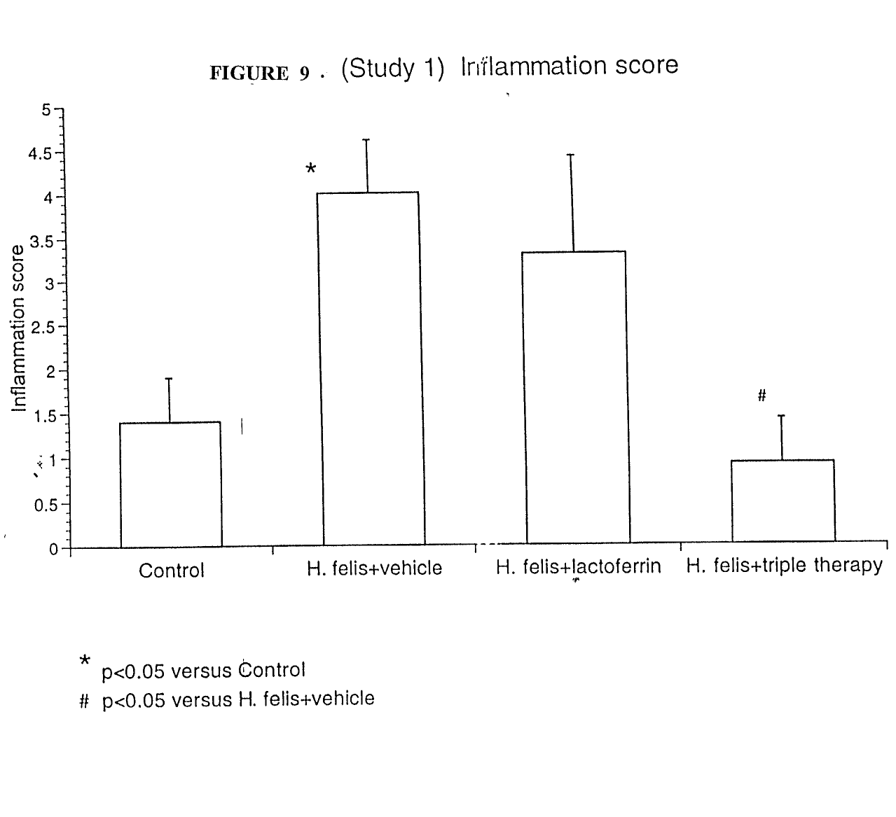

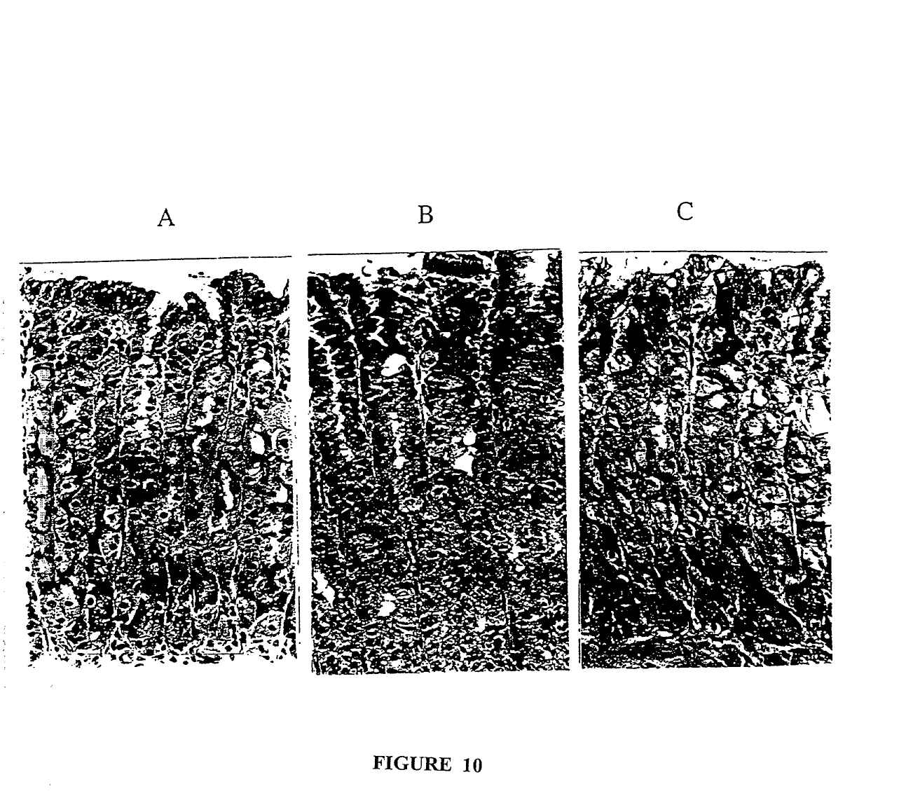

FIGS. 7 and 8 demonstrate the response over time of changes in stomach mass and gastric contact angle in control mice (uninfected), H. felis-infected mice receiving vehicle, and H. felis-infected mice receiving lactoferrin or triple therapy. It can be seen that both parameters were significantly different in infected versus uninfected mice. Further, lactoferrin treatment reversed the infection-induced changes by about 40% in each case. Examination of the inflammation score (FIG. 9) of these groups showed similar responses. That is, H. felis infection produced a significant increase in the presence of inflammatory cells in the gastric mucosa, and this increase tended to decrease by lactoferrin treatment. In addition, examination of other cell changes induced by H. felis infection revealed that the loss of parietal and chief cells seen in untreated, infected mice (FIG. 10B), compared to controls (FIG. 10A), was partially blocked by lactoferrin after 15 days of treatment (FIG. 10C). Taken together, these results suggest that this dose of orally administered human lactoferrin (100 mg/kg/day) had a significant ability to reduce damaging effects of H. felis on the mouse gastric mucosa.

Tests with omeprazole by itself revealed that omeprazole did not significantly effect stomach mass, contact angle, or inflammation score at the final day 30 time point. Further, the combination of omeprazole and lactoferrin gave the same responses on stomach mass, contact angle, and inflammation score as lactoferrin alone.

FIGS. 7, 8, and 9 also demonstrate that triple therapy was effective at reversing the changes in stomach mass, contact angle, and inflammation score that were induced by the bacteria. Therefore, this mouse model is one which can exhibit reversible, measurable changes in gastric infection by Helicobacter, and is suitable for testing pharmaceutical compounds for anti Helicobacter activity.

Study 2.

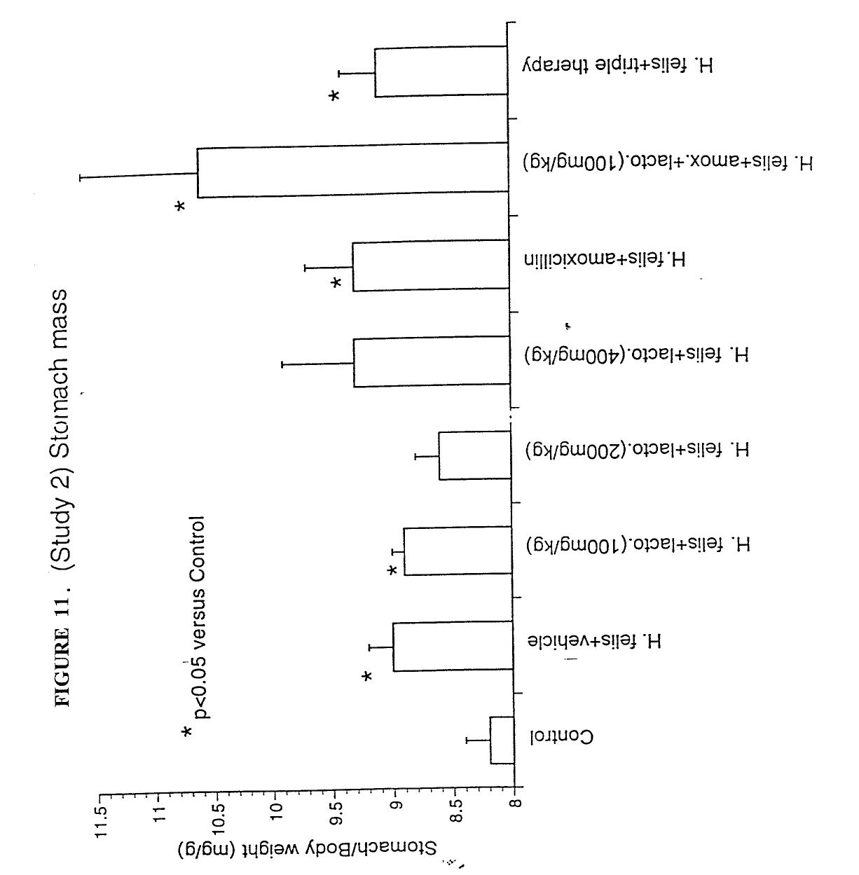

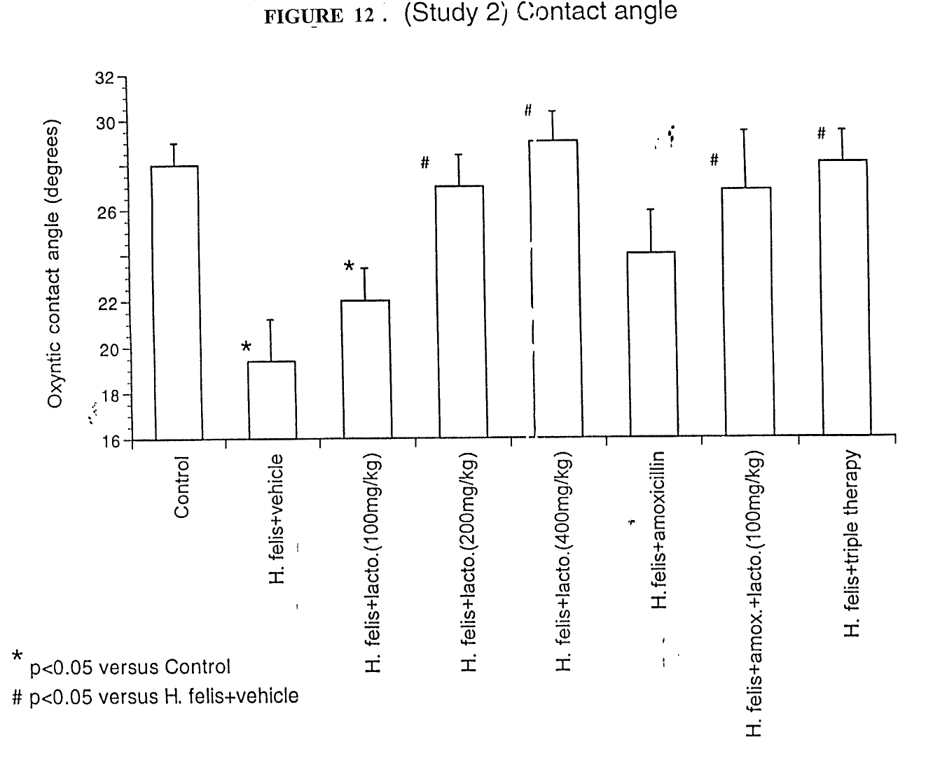

FIGS. 11 and 12 show the results from the dose-response study with lactoferrin, and the test of lactoferrin with and without amoxicillin. It can be appreciated that although the H. felis infection in Study 2 was not as severe as that seen in Study 1, a discernible and significant increase in the stomach mass and a decrease in gastric contact angle was recorded in response to infection. These changes were reversed in a dose-dependent fashion when lactoferrin was intragastrically administered at 100 and 200 mg/kg/day. It appeared that the dose of 200 mg/kg of lactoferrin gave the optimal result, in that this dose lowered the stomach mass and increased gastric contact angle so that it was not different from control mice. The highest dose of lactoferrin (400 mg/kg) also reversed contact angle changes to normal, but its effect on the stomach mass was unclear, as there was too much variability between animals to detect significant differences between it and any other group.

Treatment with amoxicillin alone (FIGS. 11 and 12) showed no effect on stomach mass and a partial reversal of contact angle changes. The combination of lactoferrin and amoxieillin revealed a complete reversal of contact angle changes and a possible enhancement of stomach mass in infected mice. The effects of lactoferrin and amoxicillin on contact angle suggest that this combination is beneficial to the gastric mucosa in infected individuals.

Conclusions

Lactoferrin treatment of H. felis-infected mice reversed bacterial-induced changes in gastric mucosal contact angle, stomach mass, gastric inflammation, and parietal cell/chief cell atrophy. These beneficial effects of lactoferrin were seen in a 2 week period, and in a dose dependent manner. The combination of lactoferrin with amoxicillin provided especially impressive effects.

### Example 9

Efficacy of Human Lactoferrin at Treating Helicobacter felis Infection in Mice.

Methods

C57BL/6 female mice were infected with viable cultures of Helicobacter felis (ATCC 49179) by oral inoculations of 0.2 mi per mouse (10¹⁰ colony forming units/ml) three times at two day intervals. Age-matched control (uninfected) mice were maintained in parallel. At each predetermined time point, the following measurements were made: Fasting body weight, pH of gastric wash, Stomach weight, Calculation of stomach/body weight ratio, Urease test of oxyntic tissue, Contact angle of oxyntic tissue, Histology of oxyntic tissue, Urease test of antral tissue, and Histology of antral tissue.

At both 2 and 4 weeks post infection the above parameters were assessed, and it was determined that the infection had sufficiently progressed by 4 weeks so that significant, measurable changes were evident. At that time drug treatments were initiated as follows.

Four groups of 8 mice each were treated daily by a single oral inoculation of 0.1 ml of rLF (1.7 mg, or −83 mg/kg/day). After 5, 9, and 15 days of treatment, mice were euthanized and samples were collected and analyzed. The additional group was treated with rLF for 15 days and days after that was assessed (30 days after treatment initiation). At each of these time points, control (uninfected) mice and H. felis-infected mice that had received vehicle daily (0.1 ml water) were also assessed. In addition, three other groups of mice were treated daily for 15 days with either omeprazole (150 mg/kg), an acid blocking drug; omeprazole plus rLF; or triple antibiotic therapy which is the standard treatment for Helicobacter infection (tetracycline, 1.5 mg; metronidazole, 0.675 mg; and bismuth subcitrate, 0.185 mg). After an additional 15 days these latter groups were also assessed.

Results

Efficacy of rLF Alone

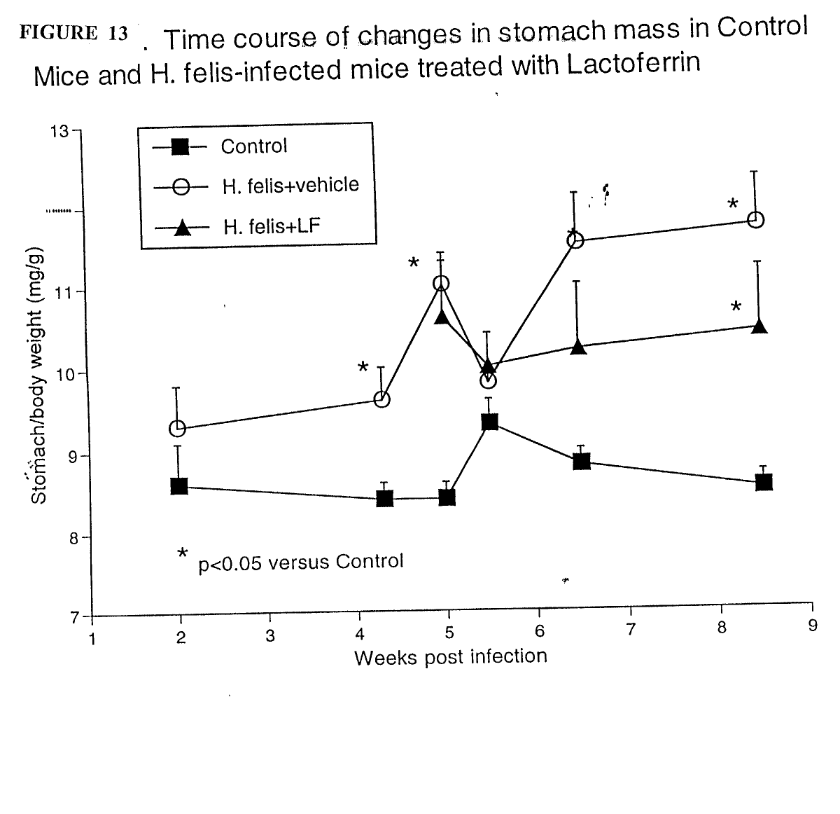

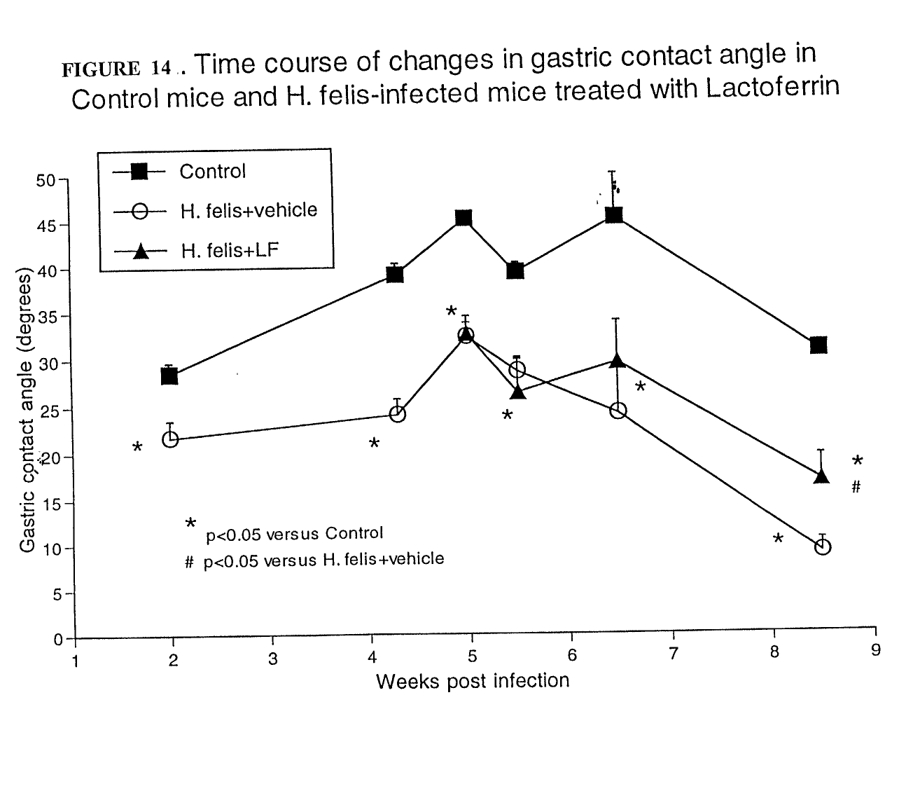

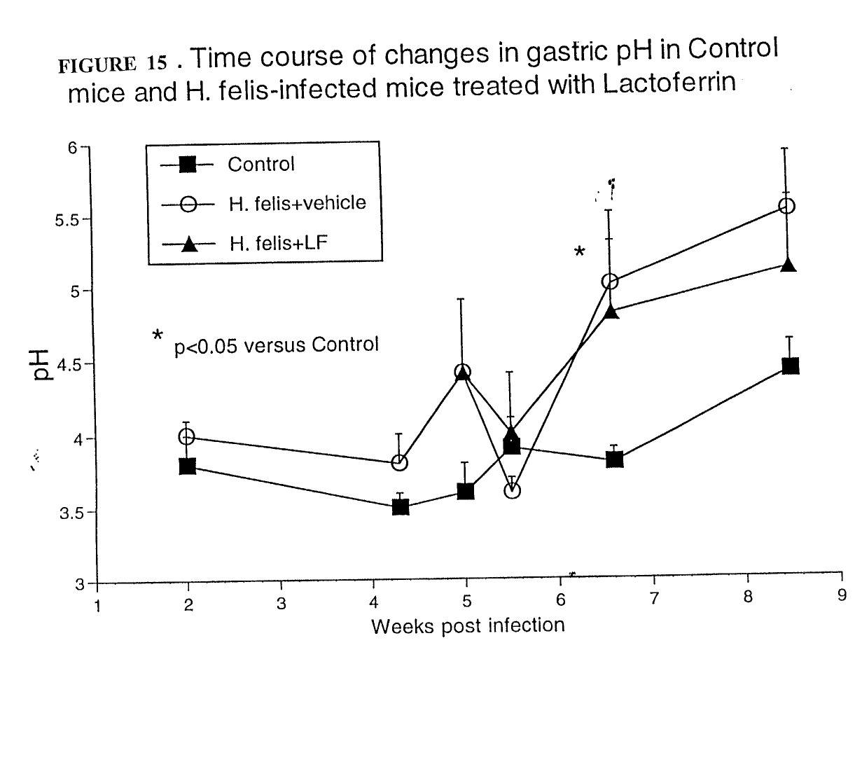

FIGS. 13, 14, and 15 illustrate the response over time of changes in stomach mass, gastric contact angle, and gastric luminal pH, respectively, for control (uninfected), H. felis-infected receiving vehicle, and H. felis-infected treated with rLF. The three groups in each figure show similar qualitative responses. That is, in each figure there is a significant difference between the control (uninfected) and the H. felis-infected mice that received vehicle. The rLF treatment of infected mice resulted in a reduction of about 40% of that difference, although it reached statistical significance for the contact angle data only (FIG. 14). These results demonstrate that orally administered rLF has the ability to reduce the progressive damaging effects of H. felis on the mouse gastric mucosa.

In Table VII it can be seen that there was urease reactivity indicating the presence of the bacteria in the oxyntic or antral tissue from all but 1 of 24 mice during the first 15 days of rLF treatment. Then after 15 days of treatment and 15 days of no treatment (day 30), 3 of 8 mice showed no urease activity (63% infection rate).

Efficacy of rLF Plus Omeprazole.

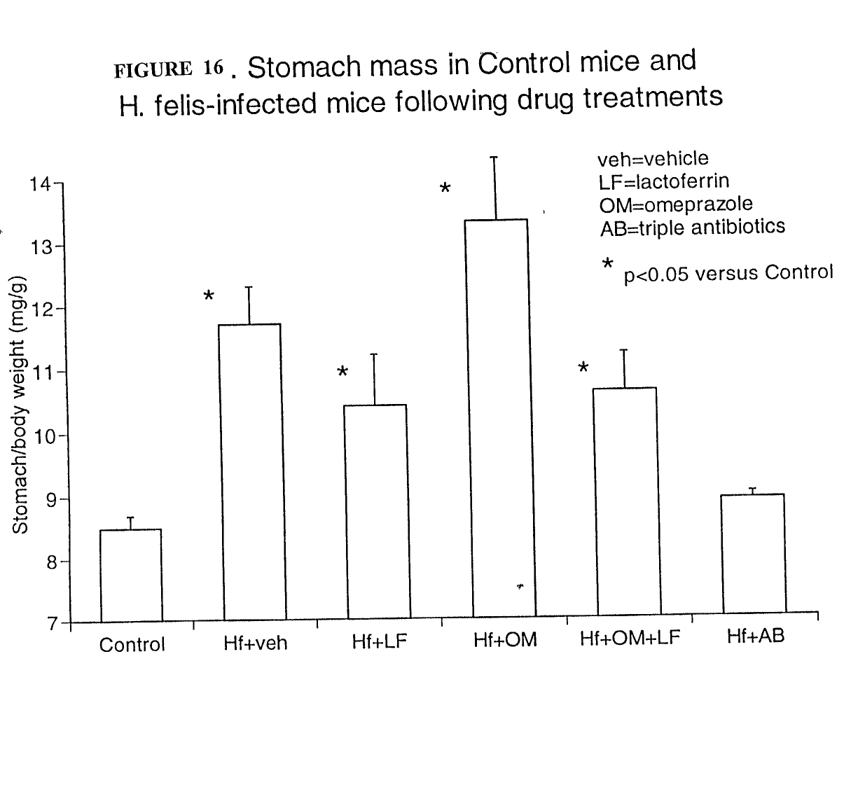

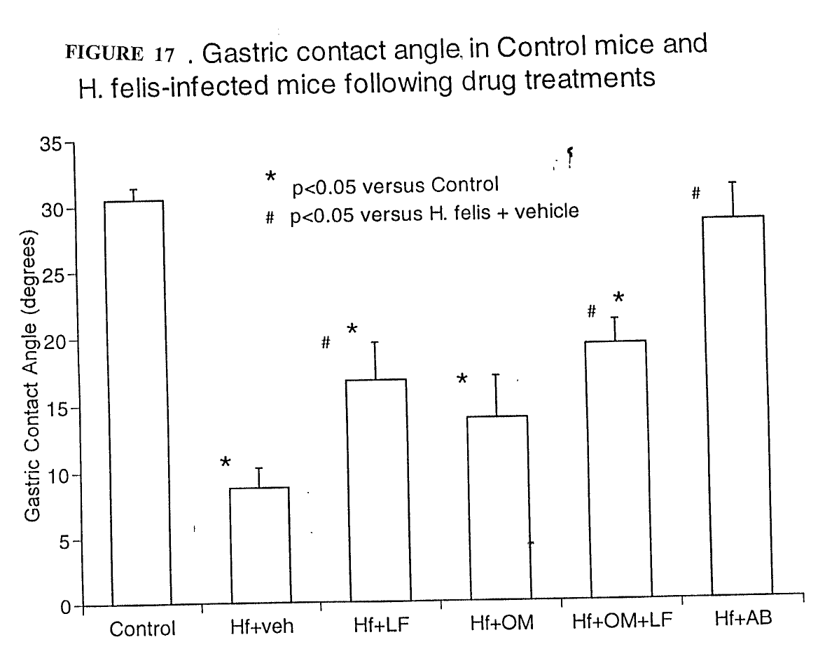

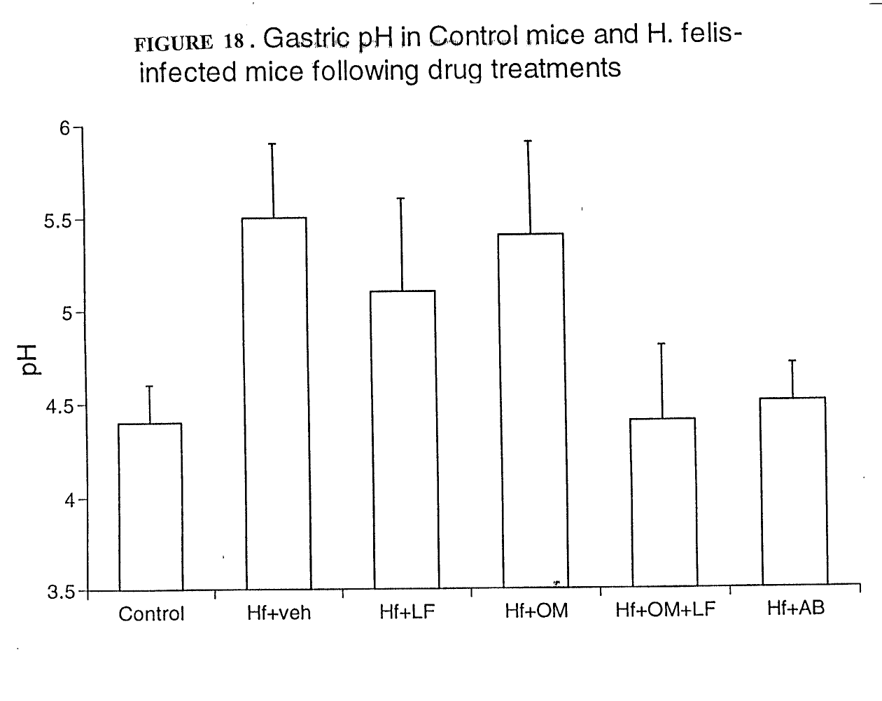

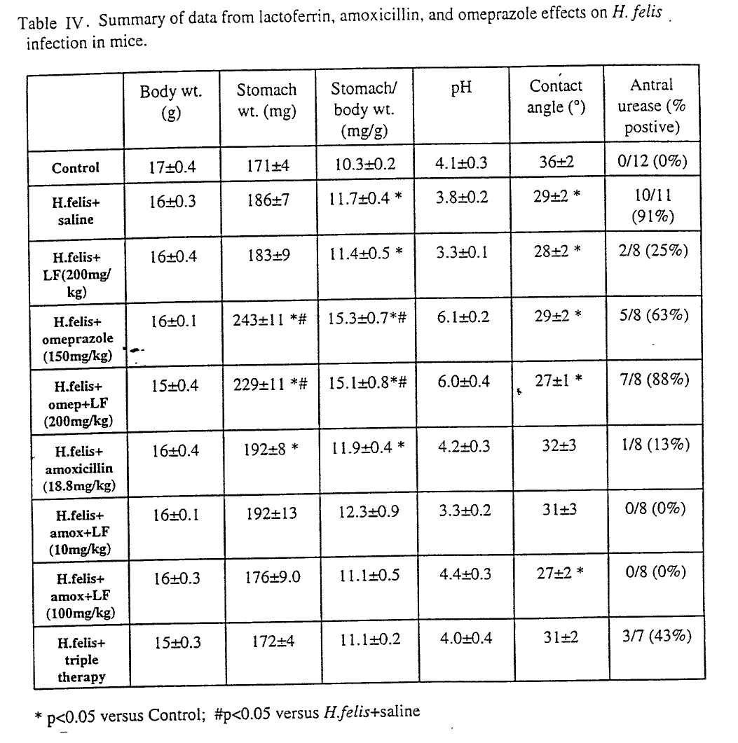

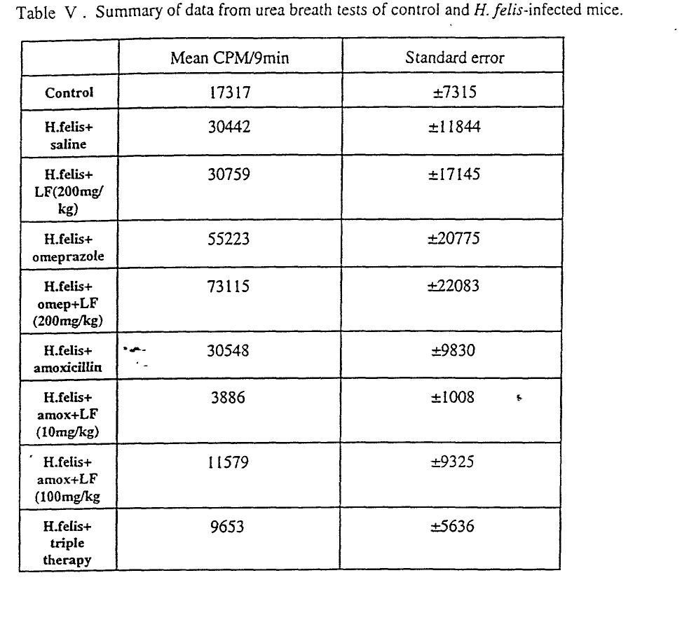

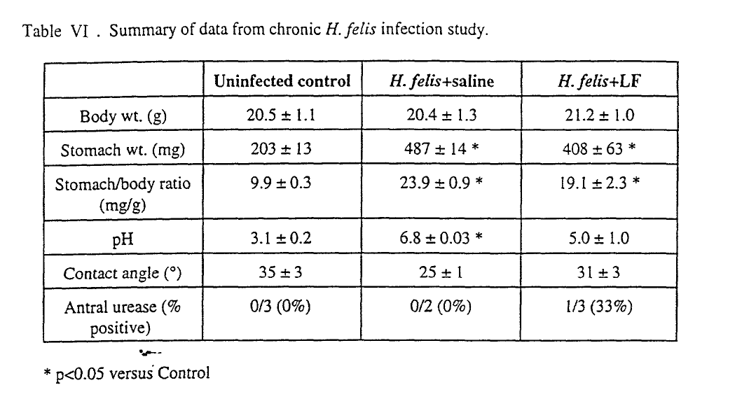

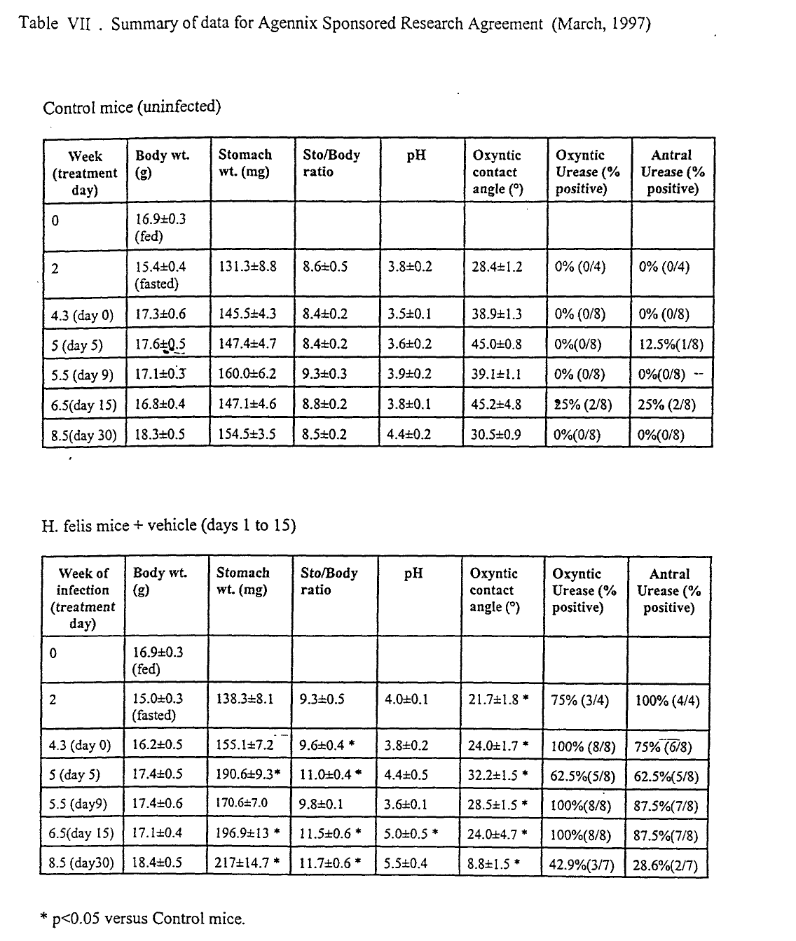

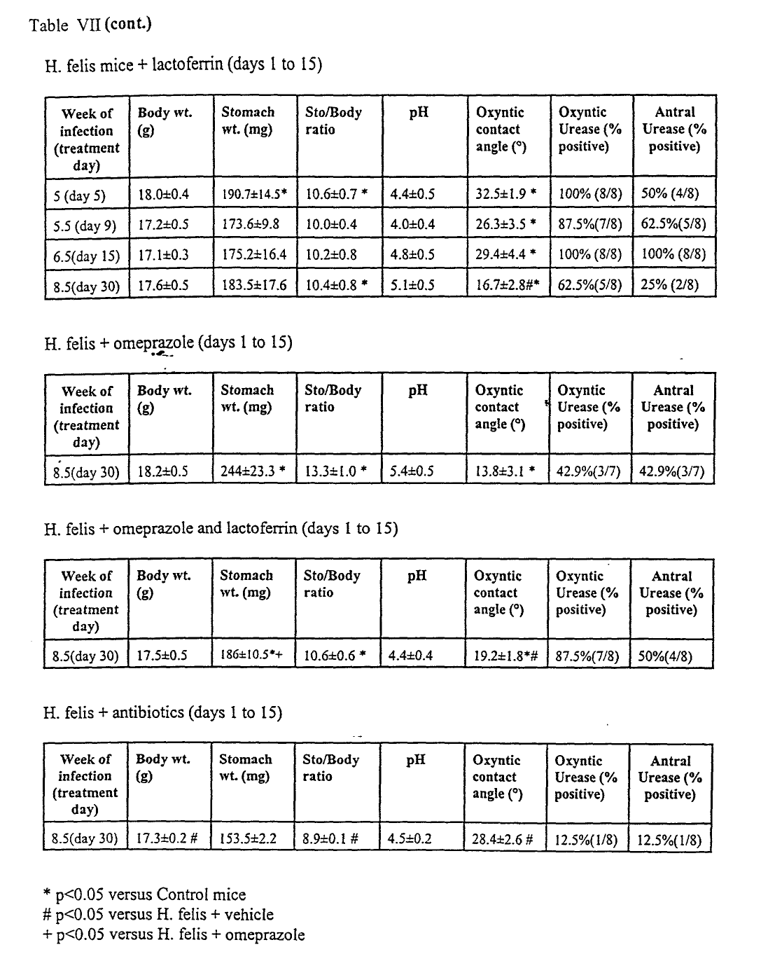

FIGS. 16, 17, and 18 show the results of omeprazole treatment alone and omeprazole plus rLF. Omeprazole alone gave variable responses. Compared to H. felis infection (+vehicle), omeprazole treatment somewhat enhanced the gastric hypertrophy (FIG. 16), reduced the reduction in contact angle (FIG. 17), and had no effect on pH (FIG. 18). In contrast, administration of rLF with omeprazole resulted in changes either identical to rLF alone (FIGS. 16 and 17), or somewhat better than rLF alone (FIG. 18).

Efficacy of Triple Antibiotic Therapy.

FIGS. 16, 17, and 18 also show that the triple antibiotic therapy was effective at reversing the changes in gastric hypertrophy, contact angle, and luminal pH that were induced by the bacteria. In all cases, the antibiotics returned parameters to control levels.

Conclusions

The H. felis-infected mouse model is suitable for showing in vivo efficacy of a drug against this gastric infection. Recombinant human lactoferrin at a dose of 83 mg/kg was effective at reducing infection parameters by around 40%.

The present invention is not to be limited in scope by the exemplified embodiments which are intended as illustrations of single aspects of the invention, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

All references cited within the body of the instant specification are hereby incorporated by reference in their entirety. In addition, the publications listed below are of interest in connection with various aspects of the invention and are incorporated herein as part of the disclosure:

Butler, T. W., Heck, L. W., Huster, W. J., Grossi, C. E. and J. C. Bar-ton, J. Imm. Meth., 108:159-170 (1988).

Conneely, O., et al., “Production of recombinant human lactoferrin”. PCT/US 93/22348, International Application No. PCT/US93/03614 filing date (Apr. 24, 1992), publication date: (Nov. 11, 1993).

Derisbourg, P., Wieruszeski, J. -M., Montreuil, J. and Spik, G., Biochem. J., 269:821-825 (1990).

Genta, R. M. and Graham, D. Y., Gastrointestinal Endoscopy 40:342-345 (1994).

Graham, D. Y., New Eng. J. Med., 328:349-350 (1993).

Graham, D. Y., Malaty, H. M., Evans, D. G., Evans, Jr., D. J., Klein, P. D. and Adam, E., Gastroent., 100:1495-1501 (1991).

Graham, D. Y., Opekun, A., Lew, G. M., Evans, Jr., D. J., Klein, P. D. and Evans, D. G., Am. J. Gastroent. 85:394-398 (1990).

Griffiths, E., “The iron-uptake systems of pathogenic bacteria,” Iron and Infection, Bullen, J. J. and Griffiths, E. (Eds.), pp. 69-138 (1987).

Kawakami, H. and Lonnerdal, B., Am. J. Physiol., 261 (Gastrointst. Liver Physiol., 24:G841-G846 (1991).

Klein, P. D., Graham, D. Y., Gaillour, A., Opekun, A. R. and O'Brian-Smith, E., The Lancet, 337:1503-1506 (1991).

Lewis, G. W., Anderson, J. G. and Smith, J. E., “Health-related aspects of the genus Aspergillus,” Aspergillus, Smith, J. E. (Ed), pp. 219-261 (1994).

Lönnerdal, B., “Immunology of milk and the neonate.” Adv. in Exp. Med and Biol., Mestecky, J., Blair, C. and Ogra, P. L (Eds.), 310:145-150 (1991).

Luqmani, Y. A., Campbell, T. A., Bennett, C., Coombes, R. C. and Paterson, I. M., Int. J. Cancer, 49:684-687 (1991).

Malaty, H. M. and Graham, D. Y., Gut, 35:742-745 (1994).

Malaty, H. M., Evans, D. G., Evans, Jr., D. J. and Graham, D. Y., Gastroent., 103:813-816 (1992).

Masson, P. L. and Heremans, J. F., Comp. Biochem. Physiol., 39B: 119-129 (1971).

Mograud, F. “H. pylori species heterogeneity,” Helicobacter pylori: Basic mechanisms to clinical cure, Hunt, R. H. and Tytgat, G. N. J. (Eds.) Proc. Int. Symp., Amelia Island, Fla., Nov. 3-6, 1993. Kluwer Acad. Pub., pp. 28-40 (1994).

Neilands, J. B., Annu. Rev. Biochem., 5-0:715-731(1981).

Soukka, T., Tenovuo, J. and Lenander-Lumikari, M., FEMS Microbiol. Lett., 90:223-228 (1992).

Spik, G., Coddeville, B, and Monreuil, J., Biochimie, 70:1459-1469 (1988).

G., Montreuil, J., Chrichton, R. R. and Mazurier, J. (Eds.) pp. 47-51 (1985).

Taylor, M. E., Fed. Reg., 58:27197-27203 (1993).

Valenti, P., Visca, P., Antonini, G. and Orsi, N., FEMS Microbiol. Lett., 33:271-275 (1986).

Ward, P. P., Lo, J. -Y., Duke, M., May, G. S., Headon, D. R. and Conneely, O. M., Biotechniglogy, 10:784-789 (1992).

Ward, P. P., May, G. S., Headon, D. R. and Corneely, O. M., Gene, 122:219-223 (1992).

1. A method for treating diseases related to enteropathogens comprising the step of administering an effective dose of a composition comprising lactoferrin.

2. The method of claim 1 wherein the disease is selected from the group consisting of histological gastritis, functional dyspepsia, duodenal ulcers, gastric ulcers, gastric cancer, chronic renal failure, HIV, pernicious anemia, Zollinger-Ellison syndrome and colonic polyps.

3. The method of claim 1 wherein the composition is administered orally.

4. The method of claim 1 wherein the composition further comprises one or more antibiotics.

5. The method of claim 4 wherein the antibiotic is amoxicillin.

6. The method of claim 1 wherein the composition further comprises one or more fatty acids.

7. The method of claim 1 wherein the composition further comprises one or more monoglycerides.

8. The method of claim 1 wherein the enteropathogen is selected from the group consisting of Shigella species, Salmonella species, Helicobacter species and E. coli.

9. The method of claim 1 wherein the lactoferrin is administered in an amount of at least about 10 mg/kg/day.

10. The method of claim 1 wherein the lactoferrin is administered in an amount of at least about 100 mg/kg/day.

11. The method of claim 1 wherein the lactoferrin is administered in an amount of at least about 200 mg/kg/day.

12. The method of claim 1 wherein the lactoferrin is administered in an amount of at least about 400 mg/kg/day.

13. A method for preventing diseases related to H. pylori comprising the step of administering a composition comprising lactoferrin.

14. The method of claim 13 wherein the disease is selected from the group comprising histological gastritis, functional dyspepsia, duodenal ulcers, gastric ulcers, gastric cancer, chronic renal failure, HIV, pernicious anemia, Zollinger-Ellison syndrome and colonic polyps.

15. The method of claim 13 wherein the composition is administered orally.

16. The method of claim 13 wherein the composition is further comprises one or more antibiotics.

17. The method of claim 16 wherein the antibiotic is amoxicillin.

18. The method of claim 13 wherein the composition further comprises one or more fatty acids.

19. The method of claim 13 wherein the composition further comprises one or more monoglycerides.

20. The method of claim 13 wherein the lactoferrin is administered in an amount of at least about 10 mg/kg/day.

21. The method of claim 13 wherein the lactoferrin is administered in an amount of at least about 100 mg/kg/day.

22. The method of claim 13 wherein the lactoferrin is administered in an amount of at least about 200 mg/kg/day.

23. The method of claim 13 wherein the lactoferrin is administered in an amount of at least about 400 mg/kg/day.

24. A composition for treating enteropathogen infection comprising lactoferrin.

25. A composition for treating enteropathogen infection comprising lactoferrin and an antibiotic.

26. The composition of claim 25 wherein the antibiotic is amoxicillin.
